(19) United States
(12) Patent Application Publication
(10) Pub. No.: US 2022/0280705 A1
(43) Pub. Date: Sep. 8, 2022

(54) METHOD AND APPARATUS EMPLOYING AN EXTRACORPOREAL BLOOD OXYGENATION CIRCUIT BLOOD FLOW CHARACTERISTIC FOR QUANTITATIVELY ASSESSING A PHYSIOLOGICAL PARAMETER OF A CONNECTED PATIENT

(71) Applicant: Transonic Systems Inc., Ithaca, NY (US)

(72) Inventor: Nikolai M. Krivitski, Ithaca, NY (US)

(21) Appl. No.: 17/681,358

(22) Filed: Feb. 25, 2022

Related U.S. Application Data
(60) Provisional application No. 63/155,063, filed on Mar. 1, 2021.

(51) Int. Cl. A61M 1/16

(52) U.S. Cl. CPC A61M 1/1698; A61M 1/1603; A61M 2205/3334; A61M 2230/04

(57) ABSTRACT

The present disclosure provides an assessment of a physiological parameter of a patient connected to an extracorporeal blood oxygenation circuit, wherein the extracorporeal blood oxygenation circuit includes a pump imparting at least a partial flow of blood through the extracorporeal blood oxygenation circuit. A controller and sensors are provided for monitoring an interaction between the pump performance and the physiological parameters of the patient connected to an extracorporeal blood oxygenation circuit. The physiological parameters of the patient include cardiac output and stroke volume. By observing the value of the withdrawn and/or delivered blood flow and/or its fluctuations or a parameter related to this blood flow and/or its fluctuations, an assessment of the physiological parameters of the patient is provided noninvasively and continuously.

### CROSS-REFERENCE TO RELATED APPLICATIONS

The present application claims the benefit of U.S. provisional patent application 63/155,063 filed Mar. 1, 2021 entitled METHOD AND APPARATUS EMPLOYING AN ARTIFICIAL CIRCUIT BLOOD FLOW CHARACTERISTIC FOR QUANTITATIVELY ASSESSING A PHYSIOLOGICAL PARAMETER OF A CONNECTED PATIENT, the entire disclosure of which is hereby incorporated by reference.

### STATEMENT REGARDING FEDERALLY SPONSORED RESEARCH OR DEVELOPMENT

Not applicable.

### THE NAMES OF THE PARTIES TO A JOINT RESEARCH AGREEMENT

Not applicable.

### REFERENCE TO A SEQUENCE LISTING

Not applicable.

### STATEMENT REGARDING PRIOR DISCLOSURES BY THE INVENTOR OR A JOINT INVENTOR

Not applicable.

### BACKGROUND OF THE INVENTION

### Field of the Invention

The present disclosure is related to a system where an artificial pump, such as an extracorporeal pump is connected to a patient's blood vessels or heart chamber to take blood out of the patient and return the blood to the patient, such as after performing a function, including increasing a pressure of the blood or imparting a treatment of the blood.

In a further configuration, the present disclosure relates to assessing a heart function or a physiological parameter corresponding to a heart function of a patient, wherein the patient is operably connected to the extracorporeal blood oxygenation circuit, wherein the assessing can be quantitative and corresponds to a measurable blood flow in the extracorporeal blood oxygenation circuit in a specific configuration, the present disclosure provides for non-invasively monitoring or quantifying a cardiac output of an ECMO patient by measuring a constant component of the blood flow in the extracorporeal blood oxygenation circuit. The present disclosure further provides for identifying an improvement (an increase) or a decline decrease) in heart function of a patient during treatment with the extracorporeal blood oxygenation circuit. It has been discovered that an increase (or improvement) in heart function, such as cardiac output or stroke volume, can be identified through a decrease in the constant component of the blood flow in the extracorporeal blood oxygenation circuit or an increase in the pulsatile component of the blood flow in the extracorporeal blood oxygenation circuit.

### Description of Related Art

Extracorporeal blood oxygenation circuits can include VV ECMO systems where the blood is removed from the central veins and returned back to the venous side of the body when the patient's lungs are malfunctioning, and VA ECMO systems where the blood is usually removed from the right atrium and returned back to the aorta when the patient's heart and lungs are malfunctioning.

In VA ECMO, a large venous (access) cannula is inserted, typically through the jugular or femoral vein, with its cannula tip located near the right atrium to withdraw blood. This cannula is then connected to an extracorporeal blood oxygenation circuit which includes a pump and a membrane oxygenator. In children and in some adults, blood is usually delivered back to the patient in the ascending aorta. Also, adults often have the blood withdrawn via a femoral venous (access) catheter with a tip close to the right atria and delivered via a femoral arterial (return) catheter with a tip in the descending aorta or even more distal from the aorta, for example in the femoral artery. In the present application, the aorta is set forth as the place of delivery, but it is understood delivery is not limited to this location. There are multiple variations of connection (sometimes with an additional third line to provide additional withdrawal or delivery at different locations), in. VA ECMO however, in each of patient blood is continuously circulated through the extracorporeal blood oxygenation circuit by being withdrawn from the patient, then circulated through an oxygenator, such as a membrane or different type of oxygenator as known in the art, where the blood is oxygenated. Then the blood is returned to the patient where the now oxygenated blood is pumped into the aorta where it mixes with blood coming from the heart.

In VV ECMO, large cannulas or a double-lumen cannula are inserted, usually through femoral and/or jugular veins with the tip located in the superior and/or inferior vena cava or, in the right atrium. These cannulas are then connected to an extracorporeal blood oxygenation circuit which includes a pump and a membrane oxygenator. Blood is usually withdrawn from one (or two locations) and delivered close to the right atrium, but there are multiple modifications. The patient's blood is continuously circulated through the extracorporeal blood oxygenation circuit, by being withdrawn from the patient, then circulated through an oxygenator, such as a membrane oxygenator, where the blood is then oxygenated. The blood is returned to the patient where the now oxygenated blood is pumped by right heart through lungs to left heart which delivers the oxygenated blood to the body tissue.

It is recognized these descriptions are general descriptions and are not limited to specific configurations or implementations of the devices.

### BRIEF SUMMARY OF THE INVENTION

Generally, the present disclosure relates to monitoring a patient having a circulatory system connected to an extracorporeal blood oxygenation circuit, wherein a pump imparts at least a partial flow of blood through the extracorporeal blood oxygenation circuit.

The present disclosure further relates to an interaction between the pump performance and the physiological parameters of the patient. The physiological parameters of the patient include, but are not limited to, heart related paraments such as cardiac output, and stroke volume, that have been found to influence the blood flow passing through the pump. From the present disclosure, by observing the value of the withdrawn and/or delivered blood flow in the extracorporeal blood oxygenation circuit and/or its fluctuations or a parameter related to this blood flow and/or its fluctuations, it is possible to assess the physiological parameters of the patient noninvasively and continuously. The present disclosure applies to any and all systems where one or more pumps are connected to a patient's cardiovascular system, including but not limited to patient treatment, procedural assist, and cardiovascular monitoring systems. The pump(s) in such a system may be but are not limited to peristaltic pumps, roller pumps, centrifugal pumps, rotary pumps, air driven pumps, gravity-driven supply/drainage branch(es), or any other kind of pump designed to support a patient or connect to a patient's cardiovascular system.

The present disclosure includes a method of assessing a patient connected to an extracorporeal blood oxygenation circuit having a blood oxygenator and a pump, the pump imparting a blood flow in the extracorporeal blood oxygenation circuit for withdrawing blood from a circulatory system of the patient and returning the blood to the circulatory system of the patient, the method including assessing a physiological parameter of the patient connected to the extracorporeal blood oxygenation circuit, the assessing corresponding to a measure of one of (i) a constant component of the blood flow in the extracorporeal blood oxygenation circuit and (ii) pulsatile component of the blood flow in the extracorporeal blood oxygenation circuit.

The present disclosure also includes an apparatus for monitoring a patient having a circulatory system connected to an extracorporeal blood oxygenation circuit, the extracorporeal blood oxygenation circuit having a blood oxygenator and a pump, wherein the pump imparts at least a partial blood flow through at least one of a portion of the circulatory system and the extracorporeal blood oxygenation circuit, the apparatus having a controller configured to receive blood flow data of a blood flow in the extracorporeal blood oxygenation circuit, the controller configured to calculate a physiological parameter of the patient based on one of a constant component of the blood flow and a pulsatile component of the blood flow in the extracorporeal blood oxygenation circuit.

### BRIEF DESCRIPTION OF THE SEVERAL VIEWS OF THE DRAWING(S)

FIG. 1 is a schematic of an extracorporeal blood oxygenation circuit as an extracorporeal membrane oxygenation (ECMO) circuit, and particularly a VA extracorporeal membrane oxygenation circuit.

FIG. 2A is a simplified hydraulic electric equivalent circuit of VA ECMO connected to the patient, for cardiopulmonary flows and ECMO flows that include pulsatile (frequency dependent) components.

FIG. 2B is a simplified hydraulic electric equivalent circuit of VA ECMO connected to the patient, for mean cardiac flow (cardiac output) and mean ECMO flow.

FIG. 3A is a schematic of centrifugal pump CP electro-hydraulic equivalent circuit connection to the patient model on VA ECMO) that can be used for in vitro experiments to investigate/measure parameters of the CP circuit, such as to establish the value of Z_{ECMO} and its constant Z_{ECMO-const} and pulsatile component Z_{ECMO-pulsatile}.

FIGS. 3B and 3C are graphs of attenuation characteristics of the centrifugal and analogous pump versus frequency of pulsation, where Points A, B, C possible location of main heart rate harmonic.

FIG. 4 is a graph of arterial pressure as a sum of constant and pulsatile components, using Fourier or other option to represent constant component.

FIG. 5 is a graph of VA ECMO arterial flow as a sum of constant and pulsatile components, using Fourier or other option to represent constant component.

FIG. 6 is a graph of blood flow in renal artery (A) and in carotid artery (B), wherein in A the blood flow in the renal artery recorded by flow probe placed on the artery during surgery and in B the carotid artery blood velocity curves recorded during ECHO examination.

FIGS. 7A and 7B illustrate the application of the superposition theorem with a zeroing of the heart pressure source component (7A) and the pump pressure source component (7B), where Z_{HEART-LUNGS} is the equivalent hydraulic impedance of cardiopulmonary system that also includes the aorta.

FIG. 8A is a graph of the venous ECMO flow as a sum of the pulsatile (1) and constant (2) components, where 8A(b) illustrates the improved cardiac function with the constant (average) ECMO flow decreasing and the pulsatile component increasing in agreement with Eq. 14 and 15; 8B is a graph of the arterial E(MO flow as a sum of the pulsatile (1) and constant (2) components, where 8B(b) illustrates the improved cardiac function with the constant (average) ECMO flow decreasing and the pulsatile component increasing in agreement with Eq. 14 and 15.

FIG. 9 is a graph showing the injected flow may only occur during the small part of the systole when the left ventricle pressure overcomes aortic pressure created also by ECMO, that is, where the internal pressure of left ventricle (LV) exceeds the pressure in the aorta and the aortic valve opens injecting stroke volume. FIG. 9 includes the creation by the left ventricle (LV) of the pulsatile component in normal patient and patient on VA ECMO, wherein (0) is the normal aortic pressure, (1) is the aortic pressure predominantly created by VA ECMO, note the aortic valve opened only a short time with small flow delivered, and (2) is the aortic pressure created by VA ECMO that is higher than the pressure that the non-well-functioning ventricle can project, there is now no pulsatile component, as the aortic valve has not opened. It is noted this case is to be avoided as there will be blood stagnation in cardiopulmonary system.

FIG. 9A is graph of arterial pressure as a sum of constant and pulsatile components, using Fourier to represent the constant component, where the heart (1) and ECMO circuit (pump) (2) contribute to the constant pressure component, and where with improved cardiac function “b” versus “a”, the contribution of the heart increases and the contribution of the ECMO circuit (pump) decreases.

FIG. 9B is a graph of arterial VA ECMO flow for different times, where an improvement of heart function is observed in “b” vs. “a” and the P_{UMP/ZSYS-const}—may be unknown. In FIG. 9B, the values of Q_{ECMO-const} are the average flows are measured and the actual pulsatile flow curves that are directly recorded.

FIG. 10 is a graph of the observed cardiac component and the breathing component of VA ECMO flow traces.

FIG. 11A is a graph of a decrease in arterial average blood flow and an increase of pulsatile component during 4 days in VA ECMO patient.

FIG. 11B is a graph of a decrease in arterial average blood flow and an increases of coefficient R % from Eq. 19a during 4 days in VA ECMO patient.

FIG. 12 is a graph of Qa and Qv in VA ECMO showing breathing component, where the positive pressure of the ventilator slows down the blood inflow into to the chest, thus decreasing the ECMO pump function which can be a possible sign of hypovolemia, the average ECMO flow is approximately 2.2 l/min, the arterial pulse flow is approximately 273 ml/min, and the venous pulse flow is approximately 285 ml/min.

FIG. 13 and the enlarged insets are graphs illustrating the variation of the magnitude of cardiac pulsation during the breathing cycle.

FIG. 13A is a graph showing the variation of the area under flow cardiac pulsation during a breathing cycle.

FIG. 14 is a graph showing the variation of the cardiac pulsatile flow with the breathing cycle in VV ECMO patient.

### DETAILED DESCRIPTION OF THE INVENTION

Generally, the present disclosure relates to when there is a pump 130 inside or outside of the body connected to a patient's arterial or venous system, or heart through a cannula or other means, wherein the pump imparts at least a partial flow of blood. For example, the present disclosure is directed to an extracorporeal blood oxygenation circuit 100 connected to a patient circulatory system 20, wherein the pump 130 associated with the extracorporeal blood oxygenation circuit imparts a flow of blood through the extracorporeal blood oxygenation circuit. Thus, the pump 130 can include, but is not limited to pump flow of a life support system having an extracorporeal blood oxygenation circuit 100 connected to the circulatory system of a patient.

The present disclosure further relates to an interaction between the pump performance and the physiological parameters of the patient fluidly connected to the pump 130. The physiological parameters of the patient include, but are not limited to, heart related paraments such as cardiac output, stroke volume, vascular resistance, intravascular blood volume, and others that will influence the blood flow passing through the pump. From the present disclosure, by observing the value of the withdrawn and/or delivered blood flow and/or its fluctuations or a parameter related to this blood flow and/or its fluctuations, it is possible to assess the physiological parameters of the patient noninvasively and continuously. The present disclosure applies to any and all systems where one or more pumps 130 are connected to the circulatory system 20 of the patient, including but not limited to patient treatment, procedural assist, and cardiovascular monitoring systems.

For purposes of description, the present system is set forth in terms of the extracorporeal blood oxygenation circuit 100, wherein the extracorporeal blood oxygenation circuit is an extracorporeal membrane oxygenation circuit, such as an ECMO circuit, including Veno-Arterial extracorporeal membrane oxygenation (VA ECMO) and Veno-Venous extracorporeal membrane oxygenation (VV ECMO). As used herein the term extracorporeal blood oxygenation circuit 100 includes an ECMO circuit and the ECMO circuit is an extracorporeal blood oxygenation circuit.

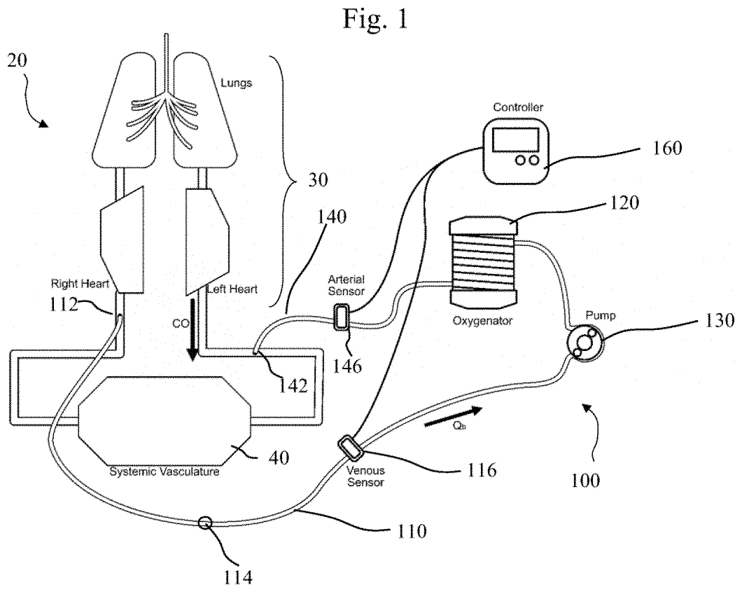

Thus, in a first configuration, a VA ECMO circuit is shown in FIG. 1. A venous (access) cannula 112 is inserted, usually through the jugular or the femoral vein, with the cannula tip located near the right atrium to withdraw blood. The cannula 112 is connected to a venous line 110 of the extracorporeal blood oxygenation circuit 100 which includes the pump 130 (typically centrifugal, but other types can be used) and a blood oxygenator 120. In children and in some adults, blood is usually delivered back to the patient in the ascending aorta. Also, adults often have the blood withdrawn via a femoral venous (access) catheter with a tip close to the right atria and delivered via a femoral arterial (return) catheter with a tip in the descending aorta or sometimes more peripheral. There are multiple variations of connection, however, in each the patient blood is continuously circulated through the extracorporeal blood oxygenation circuit 100 by being withdrawn from the patient, then circulated through the blood oxygenator 120, such as a membrane or other type oxygenator, where the blood is oxygenated. Then the blood is returned to the patient through an arterial line 140, where the now oxygenated blood is pumped into the artery or aorta where it mixes with blood coming from the heart.

Referring to FIG. 1, the extracorporeal blood oxygenation circuit 100 is shown connected to a circulatory system 20 of a patient.

The circulatory system 20 is a human (or animal) circulatory system including blood, a vascular system, and a heart. For purposes of this description, the circulatory system 20 is includes a cardiopulmonary system 30 and a systemic system 40 connecting the cardiopulmonary system 30 to the tissues of the body. Specifically, the systemic system 40 passes the blood though the vascular system (arteries, veins, and capillaries) throughout the body.

The cardiopulmonary system 30 includes the right heart, the lungs and the left heart, as well as the vascular structure connecting the right heart to the lungs, the lungs to the left heart and some portion of the aorta and large veins located between the extracorporeal blood oxygenation circuit and the right and left heart. That is, in theory the cardiopulmonary system 30 would include only the right heart, the lungs, the left heart and the vascular structure directly connecting the right heart to the lungs and the lungs to the left heart. However, in practice it is sometimes impracticable to operably connect the extracorporeal blood oxygenation circuit 100 immediately adjacent the large vein at the right heart, or immediately adjacent the aorta at the left heart. Therefore, the cardiopulmonary system 30 often includes a limited length of the veins entering the right heart and the aorta exiting the left heart. For example, the extracorporeal blood oxygenation circuit 100 can be connected to a femoral artery and femoral vein, thereby effectively extending the cardiopulmonary system 30 to such femoral artery or vein.

For cardiopulmonary and vascular systems, the term “upstream” of a given position refers to a direction against the flow of blood, and the term “downstream” of a given position is the direction of blood flow away from the given position. The “arterial” side or portion is that part in which oxygenated blood flows from the heart to the capillaries. The “venous” side or portion is that part in which blood flows from the capillaries to the heart and lungs (the cardiopulmonary system 30).

The basic components of the extracorporeal blood oxygenation circuit (or ECMO circuit) 100 for a conventional extracorporeal oxygenation machine include the access (or venous) line 110, the blood oxygenator 120 and heat exchanger (not shown), the pump 130, a return (or arterial) line 140, a sensor 116 in the venous line, a sensor 146 in the arterial line and a controller 160. For purposes of description, the access (or venous) line 110 is referred to as the access line and the return (or arterial) line 140 is referred to as the return line.

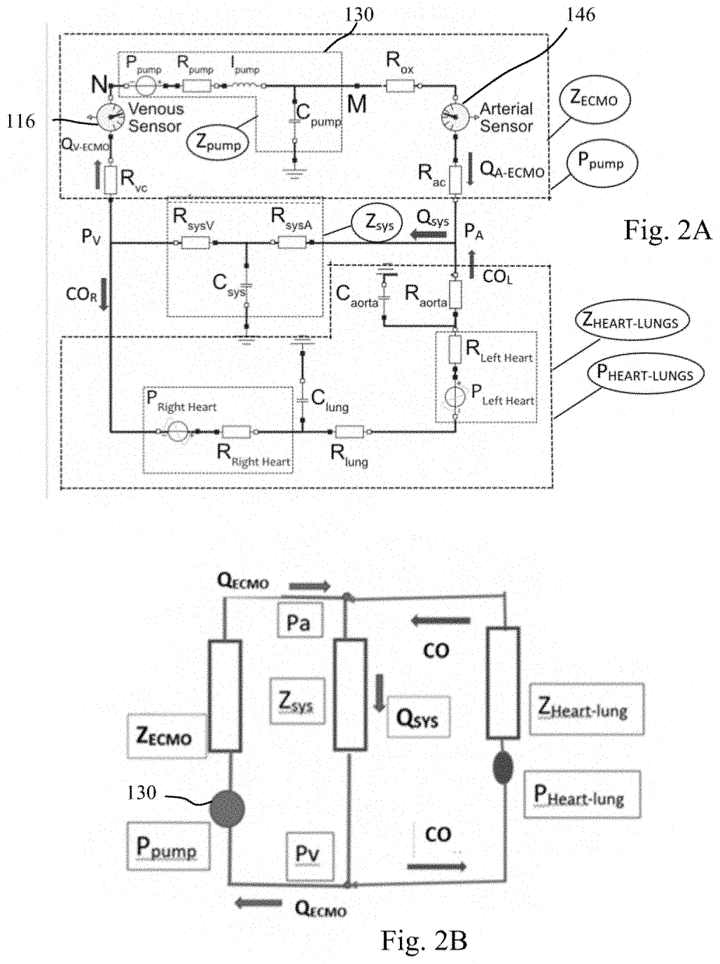

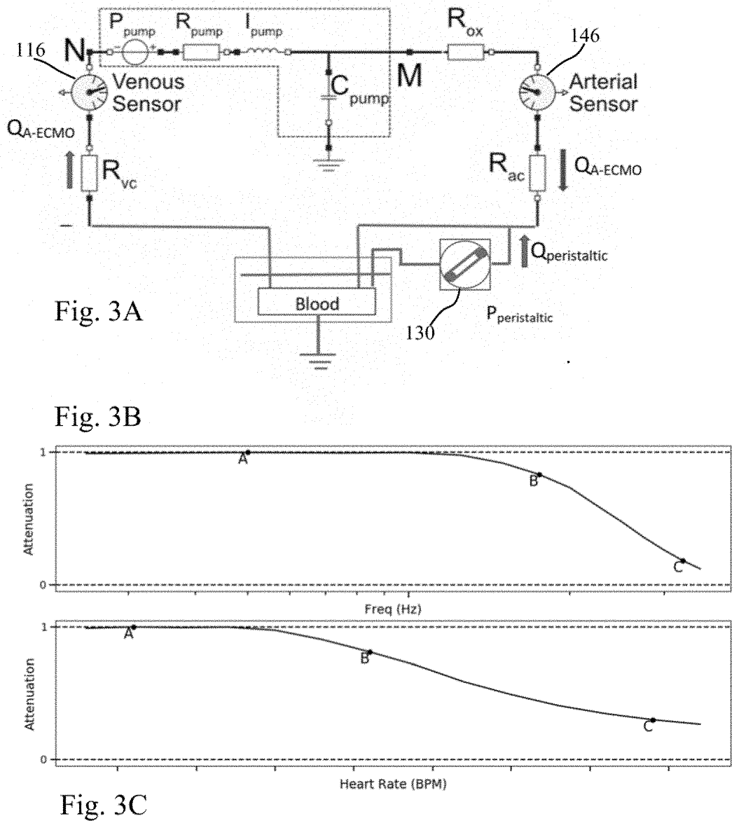

The extracorporeal blood oxygenation circuit 100 is configured to form a veno-arterial (VA) extracorporeal blood oxygenation circuit. In the veno-arterial extracorporeal blood oxygenation circuit 100, the site of the withdrawal of blood from the circulatory system 20 to the extracorporeal blood oxygenation circuit 100 is a venous portion of the circulatory system and the site of introduction of blood from the extracorporeal blood oxygenation circuit to the circulatory system is an arterial portion of the circulatory system as shown in FIGS. 2 and 3.

The site of withdrawal of blood from the circulatory system 20 to the extracorporeal blood oxygenation circuit 100 can include the inferior vena cava, the superior vena cava and/or the right atria and the site of introduction of blood from the extracorporeal blood oxygenation circuit to the circulatory system can include the aorta, a femoral artery or intermediate arterial vessels.

Thus, the VA extracorporeal blood oxygenation circuit 100 withdraws blood from the venous portion of the circulatory system 20 (including the cardiopulmonary system 30), and returns the blood to the arterial portion of the circulatory system (including the cardiopulmonary system). The withdrawn blood, can be treated while it is withdrawn, such as through gas exchange or oxygenation (ECMO) before being returned to the arterial portion of the circulatory system 20. The blood treatment can be any of a variety of treatments including, but not limited to, oxygenation (and carbon dioxide withdrawal) or merely circulation (pumping), thereby relieving the load on the heart. It is understood, the recitations of VA ECMO are not limited to any particular type of oxygenator or type of pump in the extracorporeal blood oxygenation circuit 100.

The access line 110 extends from the venous portion of the circulatory system 20, and preferably from a venous portion of the cardiopulmonary system 30. The access line 110 typically includes a venous (or access) cannula 112 providing the fluid connection to the circulatory system 20.

Depending upon the configuration of the extracorporeal blood oxygenation circuit 110 and the mechanisms for measuring the blood parameters, the access line 110 can also include or provide an indicator introduction port 114 as the site for introducing an indicator into the extracorporeal blood oxygenation circuit 100. In one configuration, the indicator introduction port 114 for introducing the dilution indicator is upstream to an inlet of the blood. oxygenator 120. In selected configurations, the introduction site 114 can be integrated into the blood oxygenator 120.

in the access line 110, the sensor 116 can be a dilution sensor for sensing passage of the indicator through the extracorporeal blood oxygenation circuit 100. The dilution sensor 116 (as well as sensor 146) can be any of a variety of sensors, and can cooperate with the particular indicator. The sensor 116 (as well as sensor 146) can measure different blood properties: such as but not limited to temperature, Doppler frequency, electrical impedance, optical properties, density, ultrasound velocity, concentration of glucose, oxygen saturation and other blood substances (any physical, electrical or chemical blood properties). It is also understood the sensor 116 can also measure the blood flow rate. Thus, in one configuration the present system includes a single blood property sensor and a single flow rate sensor. It is further contemplated that a single combined sensor for measuring flow rate and a blood parameter (property) can be used. As set forth herein, in some pumps 130, a rotational speed, RPM (rotations per minute) of the pump can be measured for providing a measurement of blood flow rate.

The return line 140 connects the extracorporeal blood oxygenation circuit 100 to an arterial portion of the circulatory system 20 and in one configuration to an arterial portion of the cardiopulmonary system 30, such as the aorta. Alternatively, the return line 140 can connect to the femoral artery. The return line 140 typically includes a return (arterial) cannula 142 providing the fluid connection to the arterial portion of the circulatory system 20.

The return line 140 can also include a sensor such as the sensor 146. The sensor 146 can be any of a variety of sensors, as set forth in the description of the sensor 116, and is typically selected to cooperate with the anticipated indicator.

It is understood the sensors 116, 146 can be located outside of the extracorporeal blood oxygenation circuit 100. That is, the sensors 116, 146 can be remotely located and measure in the extracorporeal blood oxygenation circuit 100, the changes produced in the blood from the indicator introduction or values related to the indicator introduction which can be transmitted or transferred by means of diffusion, electro-magnetic or thermal fields or by other means to the location of the sensor.

The blood oxygenator 120 can be broadly classified into bubble type oxygenators and membrane type oxygenators. The membrane type oxygenators fall under the laminate type, the coil type, and the hollow fiber type. Membrane type oxygenators offer advantages over the bubble type oxygenators as the membrane type oxygenators typically cause less blood damage, such as hemolysis, protein denaturation, and blood coagulation as compared with the bubble type oxygenators. Although the preferred configuration is set forth in terms of a membrane type oxygenator, it is understood any type of blood oxygenator 120 can be employed.

The pump 130 can be any of a variety of pumps types, including but not limited to a peristaltic or roller (or impeller or centrifugal) pump. The pump 130 may be but is not limited to peristaltic pumps, roller pumps, centrifugal pumps, rotary pumps, air driven pumps, gravity-driven supply/drainage branch(es), or any other kind of pump designed to support a patient or connect to a patient's cardiovascular system 20. The pump 130 induces a blood flow rate through the extracorporeal blood oxygenation circuit 100. Depending on the specific configuration, the pump 130 can be directly controlled at the pump or can be controlled through the controller 160 to establish a given blood flow rate in the extracorporeal blood oxygenation circuit 100. The pump 130 can be at any of a variety of locations in the extracorporeal blood oxygenation circuit 100, and is not limited to the position shown in the Figs. In one configuration, the pump 130 is a commercially available pump and can be set or adjusted to provide any of a variety of flow rates wherein the flow rate can be read by a user and/or transmitted to and read by the controller 160.

The controller 160 is typically connectable to the blood oxygenator 120, the pump 130 and the sensors 116, 146. The controller 160 can be a stand-alone device such as a personal computer, a dedicated device or embedded in one of the components, such as the pump 130 or the blood oxygenator 120. The term “controller” includes signal processors and computers, including programmed desk or laptop computers, or dedicated computers or processors. The controller 160 includes a processor programmed with the equations as set forth herein and can perform the associated calculations based on inputs from the user or connected components, wherein the controller further includes or is operably connected to a memory as known in the art. Such controllers 160 can be readily programmed to perform the recited calculations, or derivations thereof, to provide determinations of the flow rate and transforms of the flow rate data as set forth herein. The controller 160 can also perform preliminary signal conditioning such as summing one signal with another signal or portion of another signal. The controller 160 can be a stand-alone device such as a personal computer, a dedicated device or embedded in one of the components, such as the pump 130 or the blood oxygenator 120. The controller 160 can include or be operably connected to a memory, as well as an input/output device such as a touch screen or keypad or keyboard as known in the industry. Although the controller 160 is shown as connected to the first and second sensors 116, 146, the pump 130, and the blood oxygenator 120, it is understood the controller can be connected to the flow sensors, or the flow sensors and the pump, or any combination of the flow sensors, the pump, and the renal replacement therapy device. Although the controller 160 is shown as connected to the sensors 116 and 146, the pump 130 and the blood oxygenator 120, it is understood the controller can be connected to only the sensors, the sensors and the pump, or any combination of the sensors, pump and oxygenator. In one configuration, at least one of the pump 130 and the controller 160 provides for control of the pump and the flow rate of the blood through the pump, respectively. It is also understood, the controller 160 also can be connected to an oximeter, such as a pulse oximeter, to automatically collect data or oximetry data can be put manually into controller. Alternatively, the oximeter and the controller 160 can be integrated as a single unit.

The normal or forward blood flow through the extracorporeal blood oxygenation circuit 100 includes withdrawing blood through the access line 110 from the venous side circulatory system 20 (and particularly the venous portion of the cardiopulmonary circuit 30), passing the withdrawn blood through the extracorporeal blood oxygenation circuit (to treat such as oxygenate), and introducing the withdrawn (or treated or oxygenated or circulated) blood through the return line 140 into the arterial side of the circulatory system. The pump 130 thereby induces a blood flow at a known (measured) blood flow rate through the extracorporeal blood oxygenation circuit 100 from the access line 110 to the return line 140.

For purposes of the present description, the following terminology is used. Cardiac output CO is the amount of blood pumped out by the left ventricle in a given period of time (typically a 1 minute interval). The heart capacity (flow) is typically measured by cardiac output CO. The term blood flow rate means a rate of blood passage, volume per unit time. The blood flow rate is a volumetric flow rate (“flow rate”). The volumetric flow rate is a measure of a volume of liquid passing a cross-sectional area of a conduit per unit time, and may be expressed in units such as milliliters per min (ml/min) or beers per minute (l/min).

The system can include the first (venous) sensor 116 and the second (arterial) sensor 146 operatively coupled to the respective flow line and is configured to obtain flow rate data. Where the term “flow rate data” is any data from which a flow rate can be derived, assessed, or calculated, as well as any surrogate data for deriving, assessing, or calculating the flow rate. It is further contemplated that the flow rate can be the actual blood flow rate, the calculated blood flow rate, or a predicted flow rate, as well as any surrogate of the actual blood flow rate, such as but not limited to a flow velocity, or a value proportional or related to the blood flow or the velocity. The flow rate data encompasses any signals or data related to the blood flow, and particularly related to any pulsatile, varying, frequency dependent, or oscillatory component or characteristic or variation of the now, such as indicated by any signals, such as but not limited to optical signals, acoustic signals, electromagnetic signals, temperature signals and other signal that can be source of frequency analysis. The sensors 116, 146 can measure a flow characteristic or parameter to generate flow rate data, from which the now rate, or in certain configurations flow pulsation, variation, frequency change, oscillation component, or flow frequency components can be determined. Thus, the flow rate data includes any signals or data representing the flow rate or signals or data from which the flow rate, or any pulsation, variation, frequency variation, or oscillation of the flow rate, or pulsation, variation, frequency variation, or oscillation in the flow rate can be determined, or sensed, or any corresponding surrogates. For example, markers in the blood, including native or introduced particles could be used as the surrogate. Thus, the term flow rate is intended to encompass any value or measurement that corresponds to, is a surrogate of, or can represent the blood flow and especially to any pulsation, variation, frequency variation, oscillation, or a characteristic or property of the blood flow. The term “flow rate” (or “blood flow rate”) thus encompasses the volumetric flow rate as a measure of a volume of liquid passing a cross-sectional area of a conduit per unit time, and may be expressed in units of volume per unit time, typically milliliters per min (ml/min) or liters per minute (l/min), and any of its surrogates. It is understood the blood flow rate can be measured as well as calculated by any of a variety of known systems and methods. For purposes of description, measuring the flow rate encompasses obtaining or measuring the flow rate data.

Referring to FIG. 2A, a simplified hydraulic electric equivalent circuit of VA ECMO connected to the patient is shown. FIG. 2A is a simplified schematic of a hydraulic electric equivalent circuit for patient connected to VA ECMO. FIG. 2B is a simplified schematic of hydraulic electric equivalent circuit for average flows, wherein frequency dependent components are not included or in other words, the values of all the components at zero frequency, or for constant flow are included.

Non-peristaltic pumps, like centrifugal pumps (CP) or rotary pumps are often used in life supporting extracorporeal systems like ECMO, intracorporal systems, such as the Impella brand type catheter, and different types of VADs. FIG. 3A is a schematic of CP electro-hydraulic equivalent circuit connection to the patient model on VA ECMO that can be used for in vitro experiments to investigate/measure parameters of the CP circuit.

In a simplified model of patient connected to VA ECMO (FIG. 2A), the flows and pressures will be function of:
a right heart pressure P_{Right Heart},
a left heart pressure P_{Left Heart},
a constant ECMO pump pressure P_{PUMP}, and
multiple hydraulic impedances that include on the ECMO side:
an arterial cannula resistance R_{ac};
an venous cannula resistance R_{vc};
an oxygenator resistance Rₒₓ, and
an internal pump impedance Z_{PUMP} that includes:
a hydraulic capacitance of the centrifugal pump (C_{PUMP}) implying the fluid compressibility and the fluid volume,
a hydraulic inductance containing the inertial fluid lass and inertia of the centrifugal pump (L_{PUMP}), and
a hydraulic resistance of the pump (R_{PUMP}).

On the patient side, the simplified model includes:
an aorta resistance Rₐₒᵣₜₐ and aorta capacitance Cₐₒᵣₜₐ,
an internal resistance of left heart R_{Left Heart},
a resistance of lungs R_{LUNG},
a capacitance of the lungs C_{LUNG} in between the left and the right heart that mimics the transformation of pulsatile flow component in between the right heart and the left heart, while keeping the average blood flow, cardiac output CO, the same;
an internal resistance of right heart R_{Right Heart},
a systemic vascular impedance Z_{sys} that includes resistance components R_{sysV} and R_{sysA};
a capacitance component C_{SYS} that mimics the filtration (practically disappearance) of pressure/flow cardiac pulsatile component between the artery and the veins in the patient circulatory system;
Pₐ—the arterial pressure at the location of the arterial cannula tip;
P_{V}—the venous pressure at the location of the venous cannula tip;
Z_{ECMO}—the hydraulic impedance of the ECMO circuit that includes multiple hydraulic impedances of the arterial and venous cannula, of the blood oxygenator 120 and the pump 130 (FIGS. 2, 3);
Q_{A-ECMO} and Q_{V-ECMO}—the blood flows in the arterial and the venous lines;
Z_{HEART-LUNGS}—the equivalent hydraulic impedance of cardiopulmonary system that importantly also includes aorta;
P_{HEART-LUNGS}—the equivalent pressure created by the heart;
CO_{L} and CO_{R}—the blood flow in the aorta and the blood flow of the right atria respectively.

It is noted that normally (except in case of some bleeding events or other manipulations, or procedures such as infusions, etc.), the average values of Q_{A-ECMO} and Q_{V-ECMO} are the same and are equal to Q_{ECMO}, while the shape of flow curves in the arterial and venous lines of ECMO may be different.

Similarly, the average values of CO_{L}, and CU_{R} are the same and are equal to the cardiac output CO. While the shape of pulsation of blood flow in the aorta is different than the shape of pulsation of blood flow coming into the right atria during cardiac cycle.

The arterial cannula tip 142 may be located in, but not limited to, the vicinity of the ascending—descending aorta, or even more distal artery. The venous cannula tip 112 may be located in, but not limited to, the vicinity of right atrium or in more peripheral veins. The arterial-venous pressure gradient P_{AV} will then be P_{AV}=P_{A}−P_{V}.

It is recognized that essentially all parameters in model FIG. 2A, such as pressures and flows within the cardio cycle or the breathing cycle, change in time.

One of the ways to mathematically solve the model FIG. 2A, with the object to quantitatively assess the status of heart recovery, such as an increase in cardiac output, and/or an improvement of other clinic parameters, is to present pressure curves and flow curves as a sum of two components. The first component is a frequency independent constant component “const”, and the second component is a frequency dependent “pulsatile” component. It is understood the frequency independent constant, or “const,” component can be the component of the flow that is outside of any pulsatile variation, or is an average or mean of the flow (such as but not limited to pressure or flow rate) which incorporates the pulsatile component within the average, as well as an average at an average maximum or average minimum of the pulsatile component in combination with the frequency independent component. For purposes of description, the term “constant” or “const” is used to encompass each of these measures including the average or mean flows (or characteristics of such average or means). While the constant component is set forth in detail as the blood flow or the pressure in the blood flow, it is understood the constant component can be any of a variety of other flow parameters or characteristics in the blood flow in the extracorporeal blood oxygenation circuit that are influenced by or reflect a change in a function of the heart of the patient connected to the extracorporeal blood oxygenation circuit. Further, the blood flow is understood to be a quantity of unit volume per time.

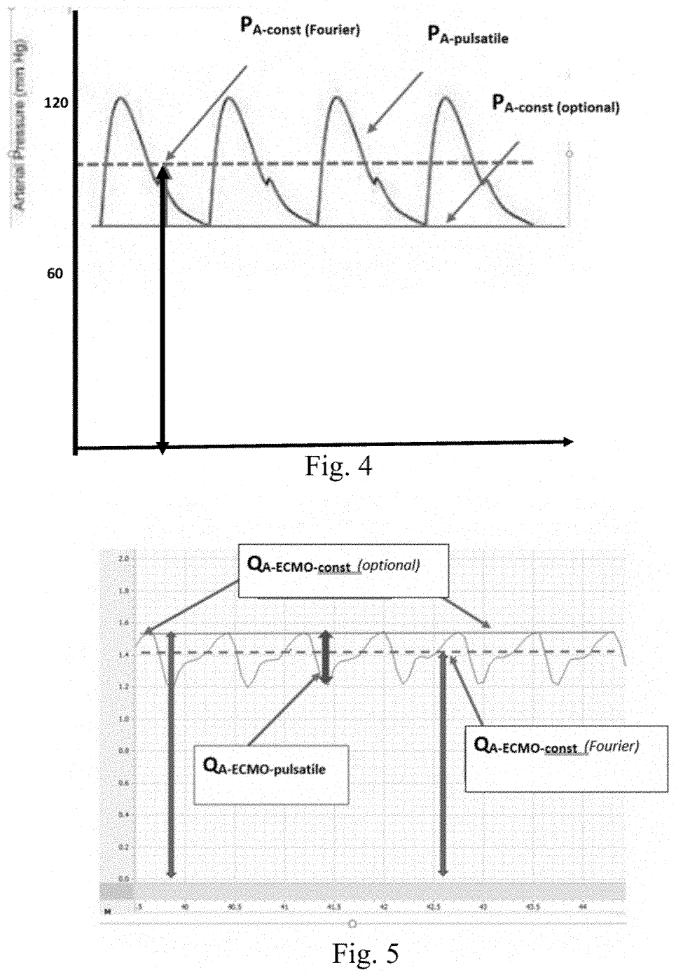

There are different ways to present the pulsatile nature of the pressure and the blood flow curves. One way (though not the only way) is using Fourier analysis. The flows and pressures of the model are presented as a sum of “const” component that can be equal to the mean value plus the sum of the various frequency components that characterize the “pulsatile” components of the parameter, the flow or the pressure. There are other ways for such presentation, for example, as optionally shown in FIG. 4 and FIG. 5. That is, the flows and pressures can be represented by various configurations of a constant component and a frequency dependent component.

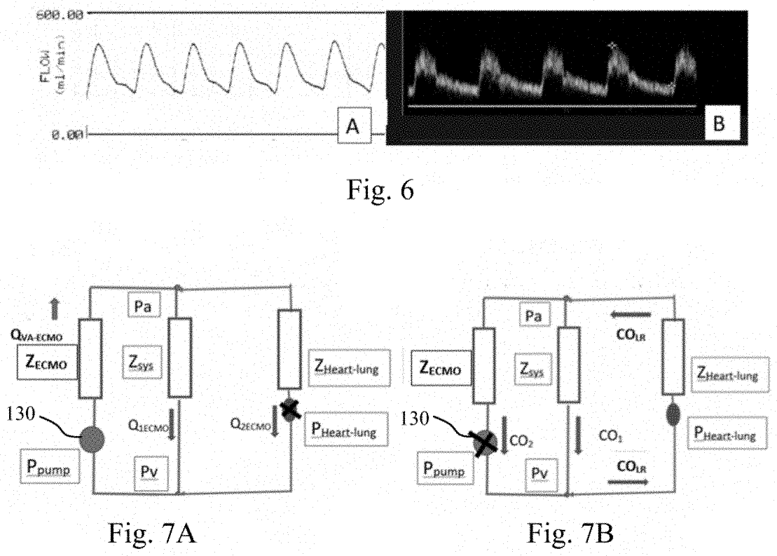

The arterial-venous pressure gradient (P_{AV}) created by the heart and by the constant ECMO blood flow, is the motive pressure that runs the blood flow in the organs (Z_{SYS}). This blood flow runs through the organs not only during systole, but during all of the cardio cycle, also during diastole (FIG. 6). Thus, this means that the constant “const” component of the blood flow also exists even without ECMO or connection to the extracorporeal blood oxygenation circuit.

It is noted that CP pump flow is produced by high frequency rotation, on the order of thousands of revolutions per minute. While theoretically speaking this pump flow is also pulsatile, however as considering that cardiac pulsation and breathing pulsation has a much lower frequency versus such pump and much larger magnitudes, for the purpose of this analysis, such PC pulsation is considered as a constant flow.

The current circuit is considered using the superposition theorem. This theorem provides a framework to calculate flows in the case of multiple pressure sources. The presentation of flow and pressures in Fourier form helps to use this theorem. The theorem provides for calculating flow in all the branches from every pressure source, while all other pressures sources assumed to be zero and then summing the flows from all sources at every branch.

In the present analysis, the first sequence of equations assumes that heart pressure source is zero (FIG. 7A) and the second sequence of equations assumes that the ECMO pressure source is zero (FIG. 7B). Note that Z_{HEART-LUNGS}—equivalent hydraulic impedance of cardiopulmonary system that importantly also includes the aorta.

For P_{Heart-lung}=0; the blood flow that generated by the ECMO pump will be distributed into parallel systemic and heart-lung circulations (FIG. 7A):

$${Q}_{VA - ECMO} = \frac{{P}_{PUMP}}{{Z}_{SYS} * \frac{{Z}_{Heart - lung}}{{Z}_{SYS} + {Z}_{Heart - lung}} + {Z}_{ECMO}} \qquad Eq . 1$$

$${Q}_{VA - ECMO} = {Q}_{1 ECMO} + {Q}_{2 ECMO} \qquad Eq . 2$$

where Q_{1ECMO} and Q_{2ECMO}—are contribution of the ECMO pump flow into systemic circulation and heart-lung circulation, respectively.

The blood flows into the branches as needed to produce same pressure gradient at point A and V:

Zsys*Q_{1ECMO}=Z_{Heart-lung}*Q_{2ECMO} Eq. 3

Or for the flow in the branches:

$$\begin{matrix}{Q}_{2 ECMO} = {Q}_{1 ECMO} * Zsys / {Z}_{Heart - lung} {Q}_{1 ECMO} * \frac{Zsys}{{Z}_{Heart - lung}} & Eq . 4 a \\ {Q}_{1 ECMO} = {Q}_{2 ECMO} * {Z}_{Heart - lung} / Zsys {Q}_{2 ECMO} * \frac{{Z}_{Heart - lung}}{Zsys} & Eq . 4 b\end{matrix}$$

Consider Eq. 2:

Q_{1ECMO}=Q_{VA-ECMO}/(1+Zsys/Z_{Heart-lung}) Eq. 5a

Q_{2ECMO}=Q_{VA-ECMO}/(1+Z_{Heart-lung}/Zsys) Eq. 5b

For Pₚᵤₘₚ=0, the blood flow generated by the heart will be distributed in the parallel circulations, the systemic circulation and the ECMO circulations (FIG. 4b):

$$\begin{matrix}{CO}_{LR} = \frac{{P}_{Heart - lung}}{\frac{{Z}_{SYS} * {Z}_{ECMO}}{{Z}_{SYS} + {Z}_{ECMO}} + {Z}_{Heart - lung}} & Eq . 6 \\ {CO}_{LR} = {CO}_{1} + {CO}_{2} & Eq . 7\end{matrix}$$

where CO₁ and CO₂—the flow contribution of Heart-lung into the systemic circulation and the ECMO circulation, respectively.

The blood flows into the branches need to produce the same pressure gradient at point A and V, thus:

Z_{ECMO}*CO₂=Zsys*Co₁ Eq. 8

Or

CO₁=CO_{LR}/(1+Zsys/Z_{ECMO}) Eq. 9

CO₂=CO_{LR}/(1+Z_{ECMO}/Zsys) Eq. 10

Now according to superposition theorem, the total flow in the ECMO and the systemic vascular resistance (SVR) and the heart branches will be the sum of flows with their signs. The solution for the average (mean) flows for FIG. 2B (see FIGS. 7A, 7B) will be:

Q_{ECMO}=Q_{VA-ECMO}−CO₂ Eq. 11a

CO=CO_{LR}−Q_{2ECMO} Eq. 12a

Q_{SVR}=Q_{1ECMO}+CO₁ Eq. 13a

The blood flow in three branches can be written consider Eq. 9 and Eq. 10 and Eq. 5a and Eq. 5b:

Q_{ECMO}=Q_{VA-ECMO}−CO_{LR}/(1+Z_{ECMO}/Zsys) Eq. 11b

CO=CO_{LR}−Q_{VA-ECMO}/(1+Z_{Heart-lung}/Zsys) Eq. 12b

Q_{SVR}=Q_{VA-ECMO}/(1+Zsys/Z_{Heart-lung})+CO_{LR}/(1+Zsys/Z_{ECMO}) Eq. 13b

Combining Eq. 11b and 12b and substituting Q_{VA-ECMO} and CO_{LR} by values from Eq. 1 and Eq. 6 one can receive for the mean values equation for cardiac output. Specifically,

Beginning with Eq. 1, Eq. 11b, & Eq. 12b

$${Q}_{VA - ECMO} = \frac{{P}_{PUMP}}{{Z}_{SYS} * \frac{{Z}_{Heart - lung}}{{Z}_{SYS} + {Z}_{Heart - lung}} + {Z}_{ECMO}} \qquad Eq . 1$$

Simplify Z_{Heart-Lung}/(Z_{Sys}+Z_{Heart-lung}) into K

$$\begin{matrix}{Q}_{VA - ECMO} = \frac{{P}_{PUMP}}{{Z}_{SYS} * K + {Z}_{ECMO}} & Eq . 1 ( Revised ) \\ {Q}_{ECMO} = {Q}_{VA - ECMO} - {CO}_{LR} / ( 1 + {Z}_{ECMO} / Zsys ) & Eq . 11 b \\ CO = {CO}_{LR} - {Q}_{VA - ECMO} / ( 1 + {Z}_{Heart - lung} / Zsys ) & Eq . 12 b\end{matrix}$$

Solving Eq. 11b for CO_{LR}:

CO_{LR}=(Q_{VA-ECMO—}Q_{ECMO})(1+Z_{ECMO}/Z_{SYS})

Plugging this result into Eq. 12b yields:

CO=(Q_{VA-ECMO}−Q_{ECMO})(1+Z_{ECMO}/Z_{SYS})−Q_{VA-ECMO}/(1+Z_{Heart-lung}/Zsys)

Factoring out Q_{VA-ECMO} and Q_{ECMO} provides:

CO=Q_{VA-ECMO}+Q_{VA-ECMO}(Z_{ECMO}/Z_{SYS})−Q_{ECMO}−Q_{ECMO}(Z_{ECMO}/Z_{SYS})−(Q_{VA-ECMO}/(1+Z_{Heart-lung}/Z_{sys}))

CO=Q_{VA-ECMO}(1+(Z_{ECMO}/Z_{SYS})−(1/1+Z_{Heart-lung}/Z_{sys})))−Q_{ECMO}(1+(Z_{ECMO}/Z_{SYS}))

CO=Q_{VA-ECMO}[1+(Z_{ECMO}/Z_{SYS})−(Z_{SYS}/(Z_{SYS}+Z_{Heart-lung}))]−Q_{ECMO}(1+(Z_{ECMO}/Z_{SYS}))

CO=Q_{VA-ECMO}[(Z_{SYS+}+Z_{ECMO})/Z_{SYS})−(Z_{SYS}/(Z_{SYS}+Z_{Heart-lung}))]−Q_{ECMO}(1+(Z_{ECMO}/Z_{SYS}))

Finding the common denominator within the brackets provides:

CO=Q_{VA-ECMO}[(Z_{SYS+}+Z_{ECMO})(Z_{SYS}+Z_{Heart-lung})/Z_{SYS}(Z_{SYS}+Z_{Heart-lung})−(Z_{SYS}*Z_{SYS}/Z_{SYS}*(Z_{SYS}+Z_{Heart-lung}))]−Q_{ECMO}(1+(Z_{ECMO}/Z_{SYS}))

Next the two parentheses are multiplied out; (where the highlighted section stays constant. It is highlighted to allow concentration on the other parameters):

CO=Q_{VA-ECMO}[((Z_{SYS+}²+Z_{SYS}Z_{Heart-lung}+Z_{ECMO}Z_{SYS}+Z_{ECMO}Z_{Heart-lung})/Z_{SYS}(Z_{SYS}+Z_{Heart-lung}))−(Z_{SYS}²/Z_{SYS}*(Z_{SYS}+Z_{Heart-lung}))]−Q_{ECMO}(1+(Z_{ECMO}/Z_{SYS}))

CO=Q_{VA-ECMO}[((Z_{SYS}²+Z_{SYS}Z_{Heart-lung}+Z_{ECMO}Z_{SYS}+Z_{ECMO}Z_{Heart-lung}−(Z_{SYS}²))/(Z_{SYS}(Z_{SYS}+Z_{Heart-lung}))]−Q_{ECMO}(1+(Z_{ECMO}/Z_{SYS}))

CO=Q_{VA-ECMO}[(Z_{SYS}Z_{Heart-lung}+Z_{ECMO}Z_{SYS}+Z_{ECMO}Z_{Heart-lung})/(Z_{SYS}(Z_{SYS}+Z_{Heart-lung}))]−Q_{ECMO}(1+(Z_{ECMO}/Z_{SYS}))

Each term is then divided by Z_{sys}:

CO=Q_{VA-ECMO}[(Z_{Heart-lung}+Z_{ECMO}+Z_{ECMO}Z_{Heart-lung}/Z_{SYS})/((Z_{SYS}+Z_{Heart-lung}))]−Q_{ECMO}(1+(Z_{ECMO}/Z_{SYS}))

Then each term is divided Z_{SYS}+Z_{Heart-lung}:

CO=Q_{VA-ECMO}[(Z_{Heart-lung}/(Z_{SYS}+Z_{Heart-lung})+Z_{ECMO}/(Z_{SYS}+Z_{Heart-lung})+(Z_{ECMO}Z_{Heart-lung}/Z_{SYS})/(Z_{SYS}+Z_{Heart-lung})]−Q_{ECMO}(1+(Z_{ECMO}/Z_{SYS}))

Then K is substituted in for Z_{Heart-Lung}/(Z_{Sys}+Z_{Heart-Lung})=K

CO=Q_{VA-ECMO}[K+Z_{ECMO}/(Z_{SYS}+Z_{Heart-lung})+(Z_{ECMO}Z_{Heart-lung}/Z_{SYS})/(Z_{SYS}+Z_{Heart-lung})]−Q_{ECMO}(1+(Z_{ECMO}/Z_{SYS}))

The Z_{ECMO} is factored out from bold terms:

CO=Q_{VA-ECMO}[(K+Z_{ECMO}(1/(Z_{SYS}+Z_{Heart-lung})+(Z_{Heart-lung}/Z_{SYS})/(Z_{SYS}+Z_{Heart-lung})]−Q_{ECMO}(1(Z_{ECMO}/Z_{SYS}))

Next a common denominator is found for the bold terms:

CO=Q_{VA-ECMO}[(K+Z_{ECMO}([Z_{SYS}+Z_{Heart-lung}]/(Z_{SYS}*(Z_{SYS}+Z_{Heart-lung}))))]−Q_{ECMO}(1+(Z_{ECMO}/Z_{SYS}))

Where Z_{SYS}+Z_{Heart-lung} now cancel to provide:

CO=Q_{VA-ECMO}[(K+Z_{ECMO}/Z_{SYS})]−Q_{ECMO}(1+(Z_{ECMO}/Z_{SYS}))

Finding another common denominator among (K+Z_{ECMO}/Z_{SYS}) provides:

CO=Q_{VA-ECMO}[((K*Z_{SYS}+Z_{ECMO})/Z_{SYS})]−Q_{ECMO}(1(Z_{ECMO}/Z_{SYS}))

Then plugging Eq. 1 (Revised) in for Q_{VA-ECMO}

$${Q}_{VA - ECMO} = \frac{{P}_{PUMP}}{{Z}_{SYS} * K + {Z}_{ECMO}} \\ CO = {P}_{PUMP} [ ( / {Z}_{SYS} ) ] - {Q}_{ECMO} ( 1 + {Z}_{ECMO} / {Z}_{SYS} ) ) \qquad Eq . 1 ( Revised )$$

Then Z_{SYS}*K Z_{ECMO} cancels out:

CO=P_{PUMP}/Z_{SYS}−Q_{ECMO}(1+(Z_{ECMO}/Z_{SYS}))

And parameters that are constant are now specified:

$$CO = \frac{{P}_{PUMP}}{{Z}_{SYS - const}} - {Q}_{ECMO - const} * ( 1 + \frac{{Z}_{ECMO - const}}{{Z}_{SYS - const}} ) \qquad Eq . 14 a$$

where CO—the cardiac output; Q_{ECMO-const}—the mean (average flow) in the VA ECMO circuit 100; Z_{SYS-const} and Z_{ECMO-const} are the hydraulic impedances for constant flow component.

Equation 14a can be rewritten to represent mean ECMO flow:

$${Q}_{ECMO - const} = \frac{{P}_{PUMP}}{{Z}_{SYS - const} + {Z}_{ECMO - const}} - CO * ( \frac{{Z}_{SYS - const}}{{Z}_{ECMO - const} + {Z}_{SYS - const}} ) \qquad Eq . 14 b$$

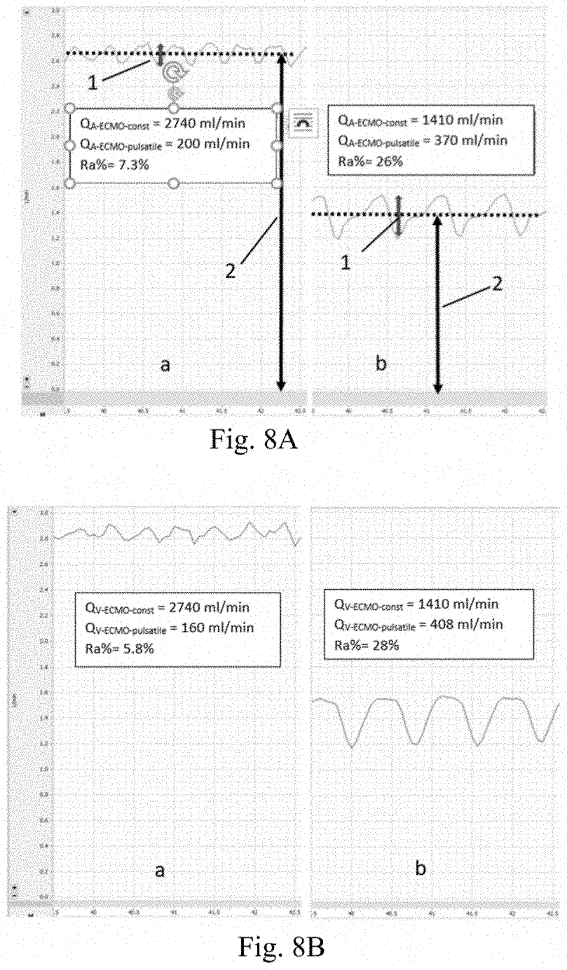

Equations 14a and 14b represent the quantitative relationship between ECMO blood flow and cardiac output. These parameters are directly related to heart recovery. With improved heart function the ECMO flow will decrease as seen in FIG. 8.

For the pulsatile component being contributed by only the heart as the single source of the pulsatile flow, then for a single frequency “f,” Eqs. 7, 8 and 10 (FIG. 7B) can be written for the pulsatile component of heart flow:

$${CO}_{LR - pulsatile ( f )} = - {Q}_{ECMO - pulsatile ( f )} * ( 1 + \frac{{Z}_{ECMO - pulsatyle ( f )}}{{Z}_{SYS - pulsatyle ( f )}} ) \qquad Eq . 15 a$$

Where CO_{LR-pulsation(f)}—represents the contribution of the pulsation component of cardiac blood flow into the ECMO flow for a chosen frequency: Z_{SYS-pulsatile(f)} and Z_{ECMO-pulsatile(f)} are the systemic and ECMO circuit hydraulic impedances at frequency “f”.

For example, one can assume that the largest contribution into the pressure and the flow pulsation component will be the heart rate (HR) f=HR. The shape of the pressure curve (FIG. 4) and the flow curve (FIG. 5) will be the sum of multiple frequency components (Fourier analysis) plus the constant component. As the equation suggests, the increases in the pulsatile component of the cardiac flow will be proportional to the increase of pulsatile component of ECMO flow, see also FIG. 8.

Equation 16 can be rewritten to represent the pulsatile component of ECMO flow:

$${Q}_{ECMO - pulsatile ( f )} = - {CO}_{LR - pulsatile ( f )} * \frac{{Z}_{SYS - pulsatyle ( f )}}{{Z}_{ECMO - pulsatyle ( f )} + {Z}_{SYS - pulsatyle ( f )}} \qquad Eq . 15 b$$

The pulsation component of the ECMO flow may be different as measured on atrial (index “A”) and venous (index “V”) side (FIG. 2A):

$$\begin{matrix}{CO}_{L - pulsatile ( f )} = - {Q}_{A - ECMO - pulsatile ( f )} * ( 1 + \frac{{Z}_{ECMO - pulsatyle ( f )}}{{Z}_{SYS - pulsatyle ( f )}} ) & Eq . 16 a \\ {CO}_{R - pulsatile ( f )} = - {Q}_{V - ECMO - pulsatile ( f )} * ( 1 + \frac{{Z}_{ECMO - pulsatyle ( f )}}{{Z}_{SYS - pulsatyle ( f )}} ) & Eq . 16 b\end{matrix}$$

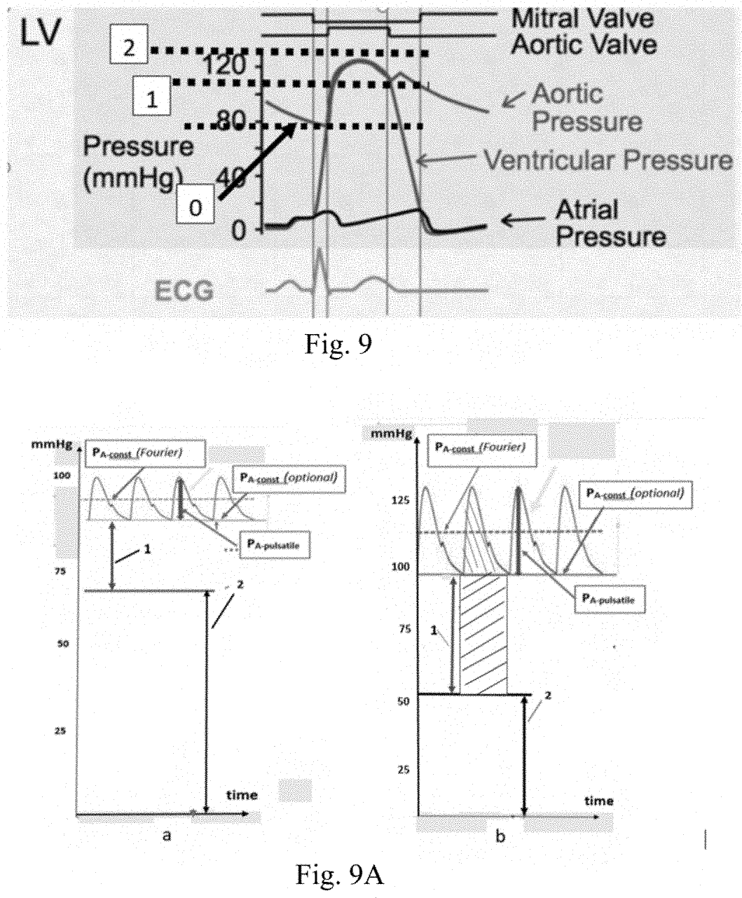

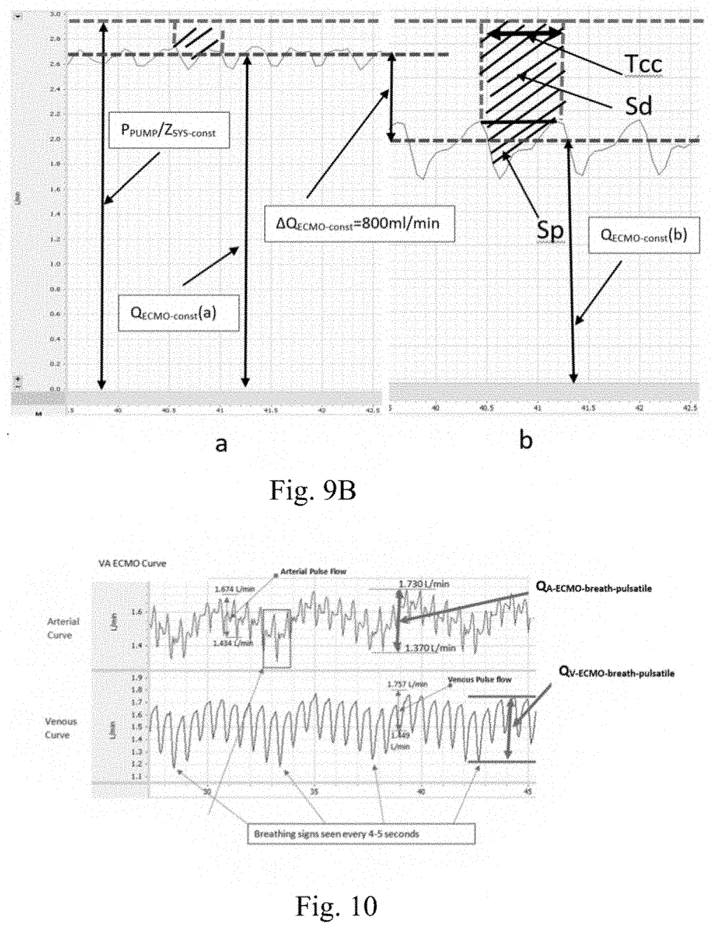

Note that the current flow model of FIGS. 2A, 2B and the following models FIG. 7A and FIG. 7B, include multiple assumptions about the complexity of the blood flow, the blood pressures in aorta and in heart chambers function. These simplifications allow one to establish relationships of measured blood flows in the extracorporeal blood oxygenation circuit (ECMO circuit) 100 and functioning parameters of cardiopulmonary system. There may be more sophisticated hemodynamic models of cardiopulmonary system, that may include modeling of valves (diodes) and other parameters. For example, modeling the left ventricle flow as zero flow during diastole (with the aortic valve dosed) and ejection flow during systole only (with the aortic valve opened). In case of VA ECMO, the injected flow may only happened during small part of the systole only when the left ventricle pressure overcomes aortic pressure created also by ECMO (FIG. 9). However, this is not a blood flow shape that is observed in arterial extracorporeal line line and organs. The stroke volume delivered into the aorta is transferred (see Cₐₒᵣₜₐ and Rₐₒᵣₜₐ) into the arterial pressure shape that is observed into blood flow in organs and in ECMO (FIGS. 4-6).

It is also understood that in addition to the Fourier analysis, the pulsatile component estimation can be calculated by measuring the magnitude of blood flow, or by measuring the area under flow curve or by other methods (FIG. 9B).

The nature of pulsatile component in VA ECMO is related (but not limited) to the pulsatile nature of the arterial pressure. The pulsatile component of the arterial pressure is related to the systole, where the internal pressure of left ventricle (LV) exceeds the pressure in the aorta and the aortic valve opens injecting stroke volume (FIG. 9). This stroke volume creates an increase of aortic pressure as blood is accumulated in the aorta. Normally, under the created pressure gradient, blood is perfused through organs (through Z_{sys}) FIG. 4. At the beginning of the VA ECMO process where a failing heart is unable to produce adequate stroke volume and keep the necessary arterial pressure, the ECMO flow delivered in vicinity of large arterial vessels is usually set to keep mean arterial pressure (MAP) at the level of around 65 mmHg to perfuse the organs. It is important to keep aortic pressure created by ECMO at the level that will allow the aortic valve to open to permit some stroke volume from the left ventricle to have blood flow through the cardiopulmonary system. If the aortic pressure created by ECMO is too high, then the malfunctioning ventricle will not be able to produce a pressure exceeding the aortic pressure to open the aortic valve (see explanation FIG. 9). This may cause blood to stagnate in the cardiopulmonary system. As the left ventricle improves, the left ventricle becomes more capable to overcome the aortic pressure created by VA ECMO. The stroke volume may increase, thereby increasing the systolic (pulsatile) component of aortic pressure (FIG. 9a), so increasing the magnitude of pulsatile component of arterial ECMO flow.

The pulsatile nature of the arterial pressure and blood flow into the organs is known to disappear as blood flows through organs via arterioles, capillaries and venulas. Thus, arterial flow pulsation is essentially eliminated by passing Z_{SYS}, and there is virtually no cardiac pulsation in large veins. In case of VA ECMO, there is direct connection through the pump circuit to transfer arterial pulsation into the veins. The arterial pulsations will be observed in the venous ECMO flow. The magnitude of these pulsations will depend on the value of Z_{ECMO} and its frequency characteristics (see pump section). Similarly, the pulsation components of the flow and the pressure in the veins including cardiac pulsation components from the right heart and breathing, which can be transferred and observed in the arterial line of the VA ECMO system (FIG. 10) will depend on the value of Z_{ECMO} and its frequency characteristics.

Eqs. 10-16 allow for quantitative assessment of CO recovery. In these equations, the values of CO and Z_{SYS} and their constant and pulsatile component are known or can be measured or calculated. Thus, these equations provide for a quantitative assessment of cardiac output CO, including a recovery or trend of the cardiac output. The values of CO and Z_{SYS} including their constant and pulsatile component are not unknowns. The values of Q_{A-ECMO-const}, Q_{A-ECMO-pulsatile}; P_{PUMP}, can be measured during ECMO. Also values of P_{PUMP} and Z_{ECMO-const} and Z_{ECMO-pulsatile} can be experimentally evaluated by bench tests (FIG. 3) for different brands of pumps and oxygenator brands and different cannulas sizes (see pump section). It is important to note that Z_{ECMO-pulsatile} is frequency dependent. The values of and Z_{ECMO-const} and Z_{ECMO-pulsatile} can also be calculated using the pressure values, if it is available:

Z_{ECMO-pulsatile(f)}=P_{AV-pulsatile(f)}/Q_{A-ECMO-pulsatile(f)} Eq. 17

Z_{ECMO-const}=(P_{PUMP}−P_{AV-const})/Q_{A-ECMO-const} Eq. 18

Note that practically P_{A} is usually measured in the radial or femoral artery that is not the location of the tip of arterial cannula. The same complications are present in measurements related to P_{V}. This moans that the measured value of P_{AV} may be not exactly the needed value in the Eq. 17 and Eq. 18, but may be related and or proportional to it or approximated by them.

Equations 10-18 can be used to assess the absolute changes in CO with time by measuring blood flow in the extracorporeal (such as ECMO)) circuit. That is, by measuring or monitoring the ECMO flow (extracorporeal blood oxygenation circuit flow) over time, the constant component or the pulsatile component, a corresponding trend of the capacity of the heart can be identified. For example, with improved cardiac function the constant (average) ECMO flow (extracorporeal blood oxygenation circuit flow) decreases and the pulsatile component increases. Thus, by monitoring the change in the ECMO flow (extracorporeal blood oxygenation circuit flow), the cardiac function can be identified as trending toward improvement or toward decline. Correspondingly, the cardiac output CO can be identified as trending toward improvement or toward decline.

The structure of Eqs. 14-16 also suggests that the heart recovery can be well represented by a ratio of pulsatile coefficient R % for the arterial and the venous extracorporeal blood oxygenation circuit (ECMO) flow (FIG. 5, FIG. 8, FIG. 10):

R_{A} %=Q_{A-ECMO-pulsatile}/Q_{A*ECMO-const}*100 Eq. 19a

R_{V} %=Q_{V-ECMO-pulsatile}/Q_{V*ECMO-const}*100 Eq. 19b

The pulsatile component of blood flow Q_{A-ECMO-pulsatile} (numerator) is an amplitude of the pulsation and is related to the stroke volume and will increases with heart recovery (FIG. 8). The denominator, Q_{A-ECMO-const}, will decrease as CO increases (FIG. 8), so the increases of R % is a very sensitive coefficient of heart recovery. Other forms of the coefficient can be applied that may include the area under pulsatile curve with different combinations.

Multiple measurements during VA ECMO

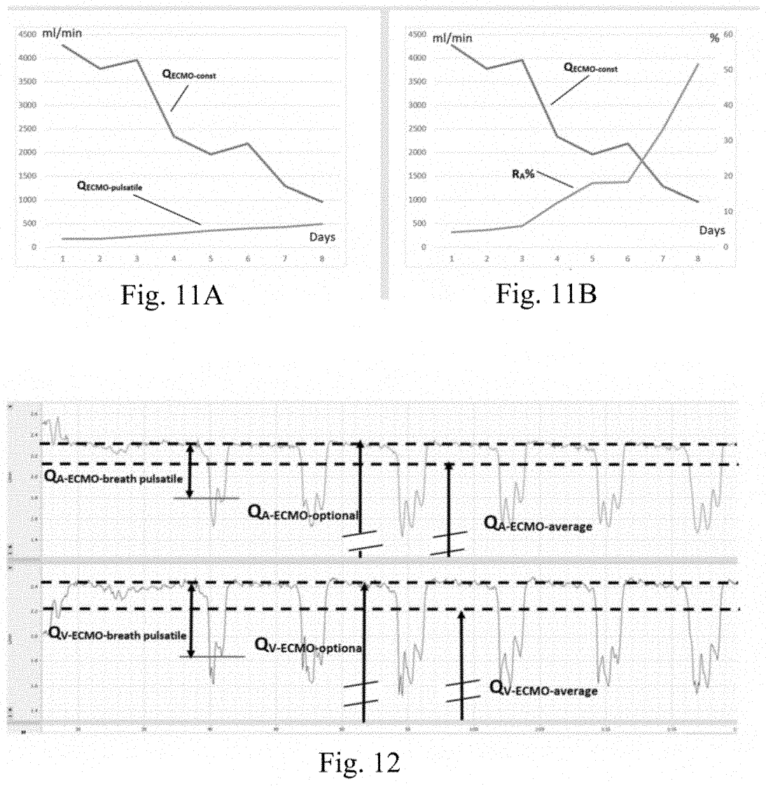

If the value of R % is very small, such as a few percent, then (see first days in FIG. 11) this means that the (dominant) organ perfusion is performed by the ECMO pump and the heart contribution is very small, thus CO≈0 and the value of initial Z_{SYS-const-initial} can be calculated from Eq. 14a:

Z_{SYS-initial}≈P_{PUMP}/Q_{ECMO-const}−Z_{ECMO-const} Eq. 20

In equation 8b, consider a small pulsatile component, then the value Q_{AM-const} can be substituted by the average blood flow. The value of Z_{ECMO-const} can be estimated from bench data (see pump section) or calculated from:

Z_{SYS-initial}≈P_{AV-const}/Q_{ECMO-const} Eq. 21

This value of initial systemic vascular impedance can be used to evaluate heart recovery, considering the value remains the same or close to this value in subsequent measurements, using Eq. 14a or as set forth in the other equations.

During the duration or VA ECMO, two measurements can be taken while the pump flow setting (P_{PUMP}) in Eq. 14a remains the same FIG. 9B. This allows for improvements in cardiac output, ΔCO, to be calculated using Eq. 14a.

$$Δ CO \approx Δ {Q}_{ECMO - const} * ( 1 + \frac{{Z}_{ECMO - const}}{{Z}_{SYS - const}} ) \qquad Eq . 14 c$$

Where ΔQ_{ECMO-const} is the decrease of the constant blood flow value (FIG. 9B).

Equation 14c can be used for estimation of absolute cardiac output changes. Systemic vascular resistance (SVR) is related to the parameter Z_{SYS}. In this analysis, it is assumed the systemic vascular resistance (SVR) stays the same or undergoes only minor changes as CO increases. It is noted that physicians try to keep the SVR of ECMO patients as close to normal range (800-1200 dynes*sec/cm⁵) as possible. From the ECMO (extracorporeal blood oxygenation circuit) side, the range of resistance of oxygenators is 800-1000 dynes*sec/cm⁵ (see table 1 in pump section). It is recognized this resistance may increase due to clotting. In addition to the resistance of the oxygenator, Z_{ECMO-cons} includes other resistance (FIG. 3). This leads to the ratio in Eq. 14c (Z_{ECMO-const}/Z_{SYS-const}) to be around one or larger. This leads to the sum in Eq. 14c to be closer to two or possibly larger. The equations 17, 18, 20, and 21 can be useful in Eq. 14c for better quantification. Practically, Eq. 14c suggests that if the ECMO flow, flow in the extracorporeal blood oxygenation circuit has dropped, for example 800 ml/min, the actual CO will have an increase larger than 800 ml/min. For example, ΔCO≈2*800=1600 ml/min. This allows the approximation of heart recovery in absolute values of ml/min from step to step, in the case of the same P_{PUMP} and the same ECMO setting.

Often at the beginning of VA ECMO, the initial pulsation is very small, which indicates that the heart is not functioning, or the CO is very small. This means P_{PUMP}/Z_{SYS-const} is close to Q_{ECMO-const(a)} (FIG. 9B(a)). When CO increases (FIG. 9B (b)), the ΔCO calculated by (Eq. 14c) will be close but will underestimate the true value of cardiac output, ΔCO≤CO.

Eq. 14c can be re-written as:

$$CO \geq Δ {Q}_{ECMO - const} * ( 1 + \frac{{Z}_{ECMO - const}}{{Z}_{SYS - const}} ) \qquad Eq . 14 d$$

Eq. 14d provides that the calculated CO underestimates the true value of the CO. If the value of CO from Eq. 14d is already at a clinically acceptable level, then the actual value of CO will be even higher, thus meaning physicians can proceed with weaning the patient from VA ECMO.

The pulsatile extracorporeal blood oxygenation circuit (ECMO) flow component also can be used for estimation of cardiac output CO. Consider the case where only arterial pulsatile flow is available, FIG. 9B(b). There is no information about P_{PUMP} or initial ECMO flow, thus the data FIG. 9B(b) is the only available data.

Normally in adults, the arterial pulse pressure (PP) value, which is the difference between systolic and diastolic pressure, PP=P_{Systole}−P_{Diastole}, is around 40-50 mm Hg, as seen in FIG. 4. The diastolic pressure is usually larger. Thus, P_{Diastole}/PP is usually larger than 1. This relationship between the diastolic and pulsatile arterial pressure components (FIG. 9A) will transfer to related extracorporeal blood oxygenation circuit (ECMO) blood flows FIG. 9B. The diastolic pressure FIG. 9A (“1”) produce a blood flow decrease of Sd (FIG. 9B(b)) during a cardiac cycle. The pulsation component of the arterial pressure will produce the arterial extracorporeal blood oxygenation circuit (ECMO) flow pulsation component Sp during a cardiac cycle (FIG. 9B(b)). When one considers the expected relationship of P_{Diastole},(PP≥1, the flow during the cardiac cycle Sd produced by diastolic component FIG. 9B(b) is expected to be at least two times larger than the flow produced by pulse pressure (Sp). Thus, the pulsatile component represents one third (⅓) of total flow produced by heart. In this case, a simple approximation of CO can be made from the observed arterial flow pulsation component in the extracorporeal blood oxygenation circuit (ECMO circuit) without any knowledge of the prior history of constant ECMO flow component and P_{PUMP}. With the prior discussion of the approximate value for the relationship between. ECMO and systemic vascular resistance of ˜1, Eq. 16a can be rewritten with the value in brackets as ˜2:

$$CO \approx 3 * \frac{{S}_{P}}{{T}_{CC}} * 2 = 6 * \frac{{S}_{P}}{{T}_{CC}} \qquad Eq . 16 c$$

Where Sp is the area under pulsatile flow component; T_{cc} is the time of cardiac cycle.

For better accuracy, the value of CO in Eq. 16c needs to be calculated for multiple cardio cycles (for example over the course of one minute) and averaged. Eq. 16c practically means that CO can be approximated as 6 times the value of the mean pulsatile flow. In considering the way the numerical coefficients were approximated, it can be assumed that the value 6 may be an underestimation. Therefore, in application, the CO approximated from Eq. 16C may underestimate the true CO. If the value of CO in Eq. 16C is already at a clinically acceptable level, then the actual value will be close to it or even higher. This means that physicians can proceed with weaning the patient from VA ECMO.

It is further contemplated that analogous concepts of the estimation of CO and its changes can be applied by intentionally increasing or decreasing the extracorporeal blood oxygenation circuit (ECMO) flow, such as by changing an RPM of the pump (P_{PUMP}) that will result in the decrease or an increase of the ECMO flow. The basic family of Eq. 14-16 and others can be used to assess the CO changes to assist in evaluating heart recovery.

Breathing flow component.

During VA ECMO at the location of the venous withdrawal cannula, the ECMO pump flow will depend on the available blood volume in the large veins (preload) and on competition with blood withdrawal by the right heart. In spontaneous breathing during the respiratory motion of the chest as the chest muscles create the negative pressure necessary to draw air into the lungs, the same negative pressure draws blood into the central veins from peripheral veins closer to the right atria. If the patient is hypovolemic, then there may be not enough blood to go into the venous cannula this during inspiration period. However, as more venous blood is available, the extracorporeal blood oxygenation circuit (ECMO circuit) flow will increase. This will show a strong modulation of flow created by the respiratory motion of the patient's chest in case of the patient being hypovolemic (FIG. 10). The flow modulation will be observed by both arterial and venous flow sensors. If the patient is on a ventilator, a high positive pressure is applied to the lungs. This high pressure on the lungs may decrease the inflow into the veins and in the case for the hypovolemic patient, the blood flow into ECMO will decrease (FIG. 12). The following coefficients can be used to evaluate patient hypovolemic status:

Breathing pulsation coefficient (FIG. 12):

R_{A-BREATH} %=Q_{A-ECMO-breath pulsatile}/Q_{A-ECMO-average}*100 Eq. 22a

R_{V-BREATH} %=Q_{V-ECMO-breath pulsatile}/Q_{V-ECMO-average}*100 Eq. 22b

Where R_{A-BREATH} % and R_{V-BREATH} %—are coefficients characterizing the relative magnitude of pulsatile component measured by atrial arid venous sensors, respectively. The value of the constant flow Q_{A-ECMO-const} is substituted by average flow Q_{A-ECMO-average}. Other forms of breathing pulsation coefficient can be used, where instead of the magnitude of pulsation, the area under the flow curve or other curve parameters can be used. Alternatively, an optional baseline can be chosen (or employed) instead of the average flow. (FIG. 12).

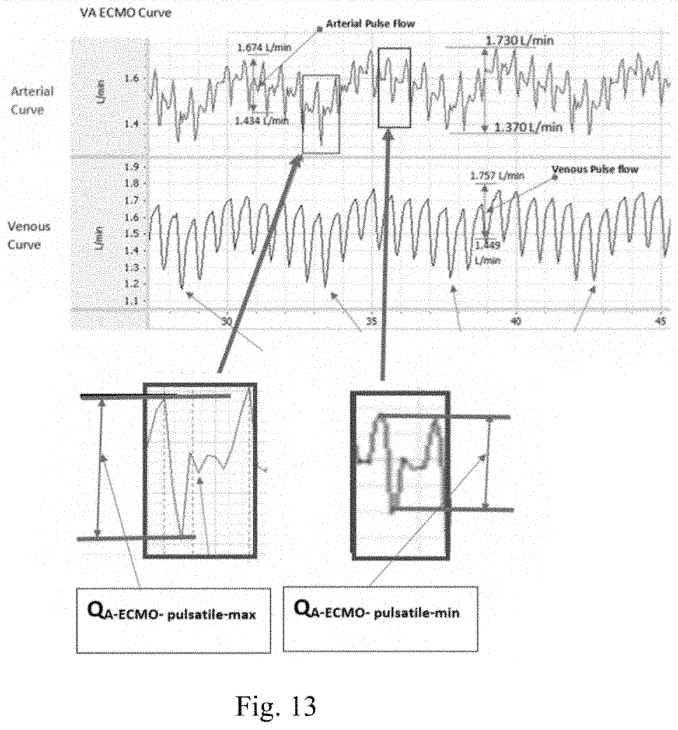

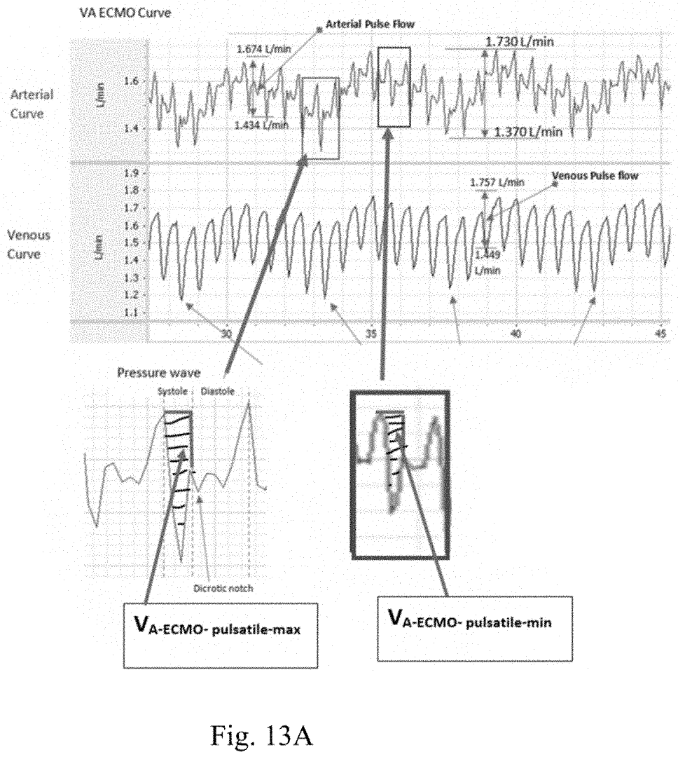

The variation of the magnitude of cardiac pulsation can be also chosen for assessment of breathing component. For example, the ratio between the larger (index “max” on FIG. 13 and the smaller (index “min”) cardiac pulsations magnitudes during breathing cycle can be used. This coefficient can also be named as a pulse flow variation.

The pulse flow variation can be calculated (FIG. 13):

$$\begin{matrix}{R}_{PV - A} \% = \frac{{Q}_{A - ECMO - pulsatile - max} - {Q}_{A - ECMO - pulsatile - min}}{{V}_{A - ECMO - pulsatile - max}} * 100 & Eq . 23 a \\ {R}_{PV - V} \% = \frac{{Q}_{V - ECMO - pulsatile - max} - {Q}_{V - ECMO - pulsatile - min}}{{Q}_{V - ECMO - pulsatile - max}} * 100 & Eq . 23 b\end{matrix}$$

where R_{PV-A} % and R_{PV-V} % are pulse flow variations recorded by the arterial sensor and the venous sensors, respectively during prolonged period of multiple cardio cycles and Q_{A-ECMO-pulsatile-max} and Q_{A-ECMO-pulsatile-min} are the largest and smallest magnitudes of cardiac flow pulsations recorded during systole during breathing cycle. Different modifications of assessment of the variation of the cardiac pulsation during the breathing cycle can be also used, for example areas under different parts of flow curves (systolic) and or area under all cardiac cycle flow curve can be used for the ratio referring to FIG. 13A.

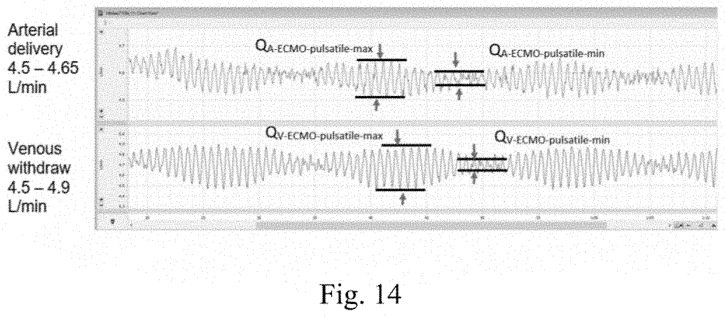

The same concept can be applied to VV ECMO (FIG. 14).

The variation of the cardiac pulsatile flow with the breathing cycle as in case of hypovolemia can also be applied to VV ECMO (FIG. 14):

$$\begin{matrix}{R}_{VV - A} \% = \frac{{Q}_{A - ECMO - pulsatile - max} - {Q}_{A - ECMO - pulsatile - min}}{{Q}_{AECMO - pulsatile - max}} * 100 & Eq . 24 a \\ {R}_{VV - V} \% = \frac{{Q}_{V - ECMO - pulsatile - max} - {Q}_{V - ECMO - pulsatile - min}}{{Q}_{V - ECMO - pulsatile - max}} * 100 & Eq . 24 b\end{matrix}$$

Where R_{VV-A} % and R_{VV-V} % are the pulse flow variation recorded by the arterial and the venous sensors, respectively in VV ECMO extracorporeal blood oxygenation circuit during prolonged period of multiple cardio cycles, Q_{A-ECMO-pulsatile-max} and Q_{A-ECMO-pulsatile-min} are the largest and the smallest amplitude of cardiac pulsation during breathing cycle recorded by the arterial sensor; and Q_{V-ECMO-pulsatile-max} and Q_{V-ECMO-pulsatile-min} are the largest and the smallest amplitude of cardiac pulsation during breathing cycle recorded by the venous sensor.

It is understood, the variety of cardiac and breathing pulsations described for VA ECMO are also applicable to VV ECMO.

Centrifugal pump, rotary and other types of pumps.

The ECMO centrifugal pump (CP) is represented in the diagram of FIG. 3 as a constant pressure source P_{PUMP}. The ECMO system includes multiple hydraulic impedances: the arterial cannula flow resistance R_{ac}; venous cannula flow resistance R_{vc}; oxygenator flow resistance Rₒₓ; and hydraulic pump impedance Zₚᵤₘₚ. The value of the hydraulic pump impedance is known to be frequency dependent mostly due to the inertia of the blood mass passing through the pump. P_{peristaltic} is the peristaltic pump with variable frequency in the range of heart and breathing frequency.

Referring to the test configuration, the CP generates the extracorporeal blood oxygenation circuit (ECMO) flow, Q_{ECMO} that creates negative pressure in the withdraw bloodline (venous line) and positive pressure in the delivery bloodline (arterial line). The pressure gradient and the resulting flow are a function ω of the radian per minute which is proportional to revolutions per minute RPM that is usually in the thousands of rotations per minute.

P_{PUMP}=K1*ω² Eq. P1

where K1 is a pump parameter. Eq. P1—is the approximate relation between RPM and the created pressure, P_{PUMP}.

For ECMO flow:

Q_{ECMO}=F(ω,Z,K2) Eq. P2

The ECMO flow in Eq. P2 is a function of multiple factors including RPM and the hydraulic impedances of the circuit Z and the construction specifics of the CP itself and K2.

The value of Z_{ECMO-const}, can be measured:

Z_{ECMO-const}=P_{PUMP}/Q_{ECMO-const} Eq. P3

where Z_{ECMO-const}=R_{ac}+R_{vc}+Rₒₓ+Z_{pump-const}, where the value of Z_{Pump-const} resistance of the pump at zero external (usually heart inflicted) frequency.

The value Z_{ECMO-const} can be tabulated for different cannula sizes and pump and oxygenator brands. Many manufacturers provide the values of oxygenator resistances for wide range of blood flows. (Table 1).

TABLE 1
Oxygenator hydraulic resistance of different adult brands.

| | Blood | | | |
|---|---|---|---|---|
| | Flow | ΔPressure | Resistance | Resistance |
| Oxygenator | (L/min) | (mmHg) | (mmHg*min/L) | (dyn*s/cm5) |
| Quadrox Adult | 3.99 | 41 | 10.3 | 822 |
| Quadrox Adult with | 3.99 | 44 | 11.1 | 889 |
| Filter | | | | |
| Quadrox Small | 3.95 | 36 | 9.2 | 738 |
| Adult & Filter | | | | |
| Terumo CAPIOX | 4.00 | 48 | 12.0 | 962 |
| FX25 | | | | |
| Medtronic Affinity | 4.02 | 50 | 12.4 | 994 |

The value of Z_{ECMO-pulsate} is frequency dependent.

The combination of Zₚᵤₘₚ with Rₒₓ will play the role of a low pass filter that will eliminate high frequency harmonics for the arterial pressure driven blood flow. In the case of the heart rate (HR) and its main harmonics being much lower than the cut of frequencies of the filter (point A), then the resistant to the pulsatile component will be close to the resistance Z_{ECMO-const}, meaning that the venous sensor can record flow pulsation caused by arterial pressure pulsation. In the case of HR and harmonics are much higher than the cut of frequency of the filter (point C) then the pressure/flow pulsation will effectively not pass through the pump and only the arterial sensors will record flow changes caused by arterial pressure. In the case of the HR and main harmonics being within the filter active range (point B), then the pulsatile flow component can be observed by venous sensors, stripped from high frequency harmonics curves may look smooth (FIG. 8). In case of the main HR harmonic being close to the resonance frequency of the extracorporeal (ECMO) circuit, one can observe an increase of amplitude of this harmonic from the arterial to the venous side. The flow pulsation on the venous side also will depend on the right heart pressures and pressures in the large veins, not only from pulsations transferred from arterial side.

To measure the frequency characteristics using the system of FIG. 3, the pulsatile pressure of peristaltic pump can be applied on the arterial side of the ECMO system and the flow can be recorded by arterial sensor Q_{A-ECMO} and by the venous sensor Q_{V-ECMO}. The ratio of the amplitude for frequency “£” spectra F(£)=Q_{V-ECMO} (£)/Q_{-ECMO} (£) can be determined (analogous to FIG. 4). Fast Fourier Transform analysis can be useful for this procedure. In some cases, Z_{ECMO-pulsate} can be defined for the main (heart rate) harmonic only. The produced pump pressure P_{PUMP} can be experimentally measured from points N and M as function of RPM. The resistance will depend on blood viscosity (hematocrit) and density.

It is understood there are other ways to calculate, or measure, the frequency characteristics of the extracorporeal blood oxygenation (ECMO) circuit 100. For example, applying a step function, such as by for example switching off the power to measure the signal that contains a very broad spectrum of frequencies and then switching the power back on. Another way to assess the frequency characteristics of extracorporeal blood oxygenation (ECMO) circuit 100 include presenting the value of Z_{Pump} as a combination of the hydraulic pump output resistance R_{PUMP} that reflects how much the output flow from the pump changes when the pump is loaded.

For purposes of the present disclosure, it is understood the hydraulic capacitance of the ECMO system (C_{ECMO}) captures all the elements in the extracorporeal (ECMO) circuit that can store and release minor fluctuations of blood volume; and the hydraulic inductance capturing the inertia of the blood mass passing through the pump L_{PUMP}.

It is also understood that the VY ECMO also may be used for assessment of heart. function such as cardiac output assessment as much as VA ECMO (see FIGS. 10, 13, 14) and not only for the breathing pulsatile component.

In summary, this or other means can be used to calculate/estimate the values of ECMO circuit parameters for its constant and pulsatile components. Per this process, the value of the parameters can be experimentally measured and tabulated for different canula sizes and oxygenators and different pumps brands and styles (like rotary pumps etc.) in blood bench experiments.

Thus, the present disclosure provides for identifying a trend in the heart function of the patient connected to the extracorporeal blood oxygenation circuit 100 having the blood oxygenator 120 and the pump 130 for imparting a blood flow in the extracorporeal blood oxygenation circuit, wherein the blood flow in the extracorporeal blood oxygenation circuit can be represented as a sum of the pulsatile components and the constant components of the measured flow, and wherein (i) a decrease in the constant component of the blood flow in the extracorporeal blood oxygenation circuit or (ii) an increase in the pulsatile component of the blood flow in the extracorporeal blood oxygenation circuit corresponds to an increase (or improvement) in the heart function, such as an increase in the cardiac output or the stroke volume of the patient heart.

While the present description is set forth in terms of VA ECMO, it is understood the present system is applicable to a VV ECMO circuit having the blood oxygenator 129 and the pump 130 withdrawing blood from the circulatory system of the patient and returning the blood to the circulatory system. It is further recognized that the term blood is used to describe the material withdrawn from the patient and the material returned to the patient, wherein the withdrawn and returned materials may not be identical. For example, the returned blood may be oxygenated relative to the withdrawn blood, and such is intended to be encompassed by the present recitation of withdrawn and returned blood.

The present disclosure further provides for an estimation of cardiac output of the patient connected to the extracorporeal blood oxygenation circuit 100. Practically, an approximation of a change in the cardiac output of the patient connected to the extracorporeal blood oxygenation circuit 100 can be made corresponding to approximately twice the change in the constant component of the blood flow in the extracorporeal blood oxygenation circuit. For example, if the constant component of the blood flow in the extracorporeal blood oxygenation circuit 100 decreases by 400 ml/min, then the change cardiac output can be estimated as 2×400 ml/min or 800 ml/min increase. Generally, the change in cardiac output of the patient can be estimated by multiplying the change in the constant component of the blood flow in the extracorporeal blood oxygenation circuit by a constant. In select configurations, the constant can be between 1.5 and 2.5, or between 1.75 and 2.25. In further configurations, a value of 2 can be used as the constant.

The present disclosure can be employed with extracorporeal blood oxygenation circuits 100 connected to a circulatory system 20 of a patient, wherein the extracorporeal blood oxygenation circuit includes the pump 130 for imparting a flow of blood from the patient, through the extracorporeal blood oxygenation circuit and back to the patient, and the blood oxygenator 120. Generally, the present disclosure identifies and quantifies a relationship between a physiological parameter that varies, or influences a blood flow through the extracorporeal blood oxygenation circuit 100 and a functioning of the heart. The functioning of the heart can be expressed through any of a variety of para cents including, cardiac output CO, stroke volume SV, SVR. The present system can be used for identifying trends in functioning of the heart, as well as estimations and calculations of absolute values of heart functioning, such as but not limited to cardiac output CO, stroke volume SV, and SVR.

The present disclosure also provides a model that characteristics of the model from Eqs. 14, 15 and 19:

R %=Q_{ECMO-p}/Q_{ECMO-p}/Q_{ECMO}*100%=CO_{P}/(P_{PUMP}/Z_{SYS}−CO)

where CO and COₚ are, respectively, the average and the pulsatile component of the blood flow in the aorta that perfuses the organs, Q_{ECMO} and Q_{ECMO-p} are, respectively, the average and the pulsatile components of arterial ECMO blood flow, P_{PUMP} is the pump pressure, Z_{SYS} and Z_{SUS-p} are, respectively, the systemic vascular resistance to constant and the pulsatile component of blood flow. That is, instead of Q_{ECMO-p} and Q_{ECMO} in R in Eq. 19a, their values from 14 b and 15 b are employed.

It is understood in the extracorporeal blood oxygenation circuit 100, the pump 130 can be any type of pump used to impart at least a partial flow of blood through the circuit. The extracorporeal blood oxygenation circuit 100 can be used to impart any of a variety of treatments of the blood including but not limited to dialysis, oxygenation as well as transfer or pumping.

It is further contemplated that the recited blood flow can be any surrogate of the actual blood flow, such as but not limited to a flow velocity, or a value proportional or related to the blood flow or the velocity. For example, markers in the blood, including native or introduced particles could be used as the surrogate. Thus, the term blood flow is intended to encompass any value or measurement that corresponds to or can represent the blood flow or a characteristic or a property of the blood flow.

Further, it is understood that indicator dilution can be used to measure blood flow, wherein the indicator generates a dilution curve of any change in the physical or chemical blood property can be sensed, identified or measured. These physical properties include, but not limited to thermal properties, optical properties, electromagnetic properties, blood density and others. Blood concentration of ions, gas concentrations, protein concentrations, radio isotopes and other concentration changes can be introduced in the blood to generate dilution curves, and hence the compound dilution curve. The dilution curves may be expressed in % concentration units or no units or in other units like volts or in units of blood parameters that were changed or in units of substances that were injected like isotopes and others.

Thus, the indicator includes but is not limited to: blood hematocrit, blood protein, sodium chloride, dyes, blood urea nitrogen, a change in ultrafiltration rate, glucose, lithium chloride and radioactive isotopes and microspheres, or any other measurable blood property or parameter. An injectable indicator may be any of the known indicators including saline, electrolytes, water and temperature gradient indicator bolus. Preferably, the indicator is non-toxic with respect to the patient and non-reactive with the material of the system. The indicator may be any substance that will change a blood chemical or physical characteristic. The indicator may be a physically injected material such as saline. Alternatively, the indicator may be by manipulating blood properties without introduction of an indicator volume, such as by heating or cooling the blood or changing electromagnetic blood properties or chemical blood properties.

A sensor is employed to detect passage of the indicator and thus measures, identified or monitors a blood parameter or property, and particularly variations of the blood parameter or property. Thus, the sensor is capable of sensing a change is a blood property, parameter or characteristic. For purposes of the disclosure, the sensor can be referred to as a dilution sensor, but this label is not intended to limit the scope of available sensors. Ultrasound velocity sensors as well as temperature sensors and optical sensors, density or electrical impedance sensors, chemical or physical sensors may be used to detect changes in blood parameters. It is understood that other sensors that can detect blood property changes may be employed. The operating parameters of the particular system will substantially dictate the specific design characteristics of the dilution sensor, such as the particular sound velocity sensor. For example, if a thermal sensor is employed, the thermal sensor can be any sensor that can measure temperature, for example, but not limited to thermistor, thermocouple, electrical impedance sensor (electrical impedance of blood changes with temperature change), ultrasound velocity sensor (blood ultrasound velocity changes with temperature), blood density sensor and analogous devices. Therefore, any type of optical sensor, impedance, resistance or electrical sensors which measure a changeable blood parameter such as the sound or ultrasound velocity in blood can be calibrated. Electrical resistance of the blood cart be measured, as the resistance depends on the volume of red blood cells (hematocrit). Calibration can be provided for ultrasound velocity sensors, as well as temperature sensors and optical density, density or electrical impedance sensors can be used to detect changes in blood parameters.

Thus, the present disclosure provides a method for assessing a physiological parameter of the patient connected to the extracorporeal blood oxygenation circuit 100, wherein the assessing the physiological parameter includes (a) identifying a pump pressure of the pump 130 in the extracorporeal blood oxygenation circuit, a hydraulic resistance of the extracorporeal blood oxygenation circuit, a hydraulic resistance of the circulatory system, and the blood flow through the extracorporeal blood oxygenation circuit; and (b) quantifying the cardiac output of the patient corresponding to the identified pump pressure, the hydraulic resistance of the extracorporeal blood oxygenation circuit, the hydraulic resistance of the circulatory system, and the blood flow through the extracorporeal blood oxygenation circuit.

The present disclosure also provides the controller 160 can be configured to calculate a quantitative relationship among a set of terms including (i) a mean blood flow in the extracorporeal blood oxygenation circuit 100 as adjusted by a factor of a circulatory system impedance and an extracorporeal blood oxygenation circuit impedance, (ii) a pump pressure adjusted by a factor of the circulatory impedance, and a (iii) cardiac output of the patient. It is contemplated the controller 160 can be further configured to determine a quantitative value of a cardiac output of the patient, wherein the quantitative value corresponds to a term of a mean pump pressure adjusted by a factor of a circulatory system impedance less the constant blood flow in the extracorporeal blood oxygenation circuit adjusted by a factor of the circulatory system impedance and the extracorporeal blood oxygenation circuit impedance. In addition, the controller 160 can be configured to adjust a value of the constant component of the blood flow in the extracorporeal blood oxygenation circuit 100 by a ratio of the hydraulic resistance of the circulatory system and a hydraulic resistance of the extracorporeal blood oxygenation circuit.

In the present system, assessing the physiological parameter includes estimating a cardiac output of the patient by multiplying a mean pulsatile flow in the extracorporeal blood oxygenation circuit 100 at a first time by a fixed number to provide a first estimate of the cardiac output of the patient at the first time. The fixed number is configured to provide an estimate that is less than an actual cardiac output at the first time. The system further contemplates multiplying the mean pulsatile flow in the extracorporeal blood oxygenation circuit 100 at a second time by the fixed number to provide a second estimate of the cardiac output of the patient at the second time. The fixed number is selected to provide at least one of the first estimate and the second estimate being less than an actual cardiac output of the patient at the first time and the second time, respectively. In one configuration, the fixed number is between 4.5 and 7.5; in another configuration the fixed number is between 5 and 7; in a further configuration the fixed number is between 5.5 and 6.5; and in one configuration the fixed number is 6. Thus, the system can provide a quantitative assessment of the physiological parameter.

This disclosure has been described in detail with particular reference to an embodiment, but it will be understood that variations and modifications can be effected within the spirit and scope of the disclosure. The presently disclosed embodiments are therefore considered in all respects to be illustrative and not restrictive. The scope of the invention is indicated by the appended claims, and all changes that come within the meaning and range of equivalents thereof are intended to be embraced therein.

What is claimed:

1. A method of assessing a patient connected to an extracorporeal blood oxygenation circuit having a blood oxygenator and a pump, the pump imparting a blood flow in the extracorporeal blood oxygenation circuit for withdrawing blood from a circulatory system of the patient and returning the blood to the circulatory system of the patient, the method comprising:
(a) assessing a physiological parameter of the patient connected to the extracorporeal blood oxygenation circuit, the assessing corresponding to a measure of one of (i) a constant component of the blood flow in the extracorporeal blood oxygenation circuit and (ii) a pulsatile component of the blood flow in the extracorporeal blood oxygenation circuit.

2. The method of claim 1, wherein assessing the physiological parameter includes assessing a change in the physiological parameter.

3. The method of claim 1, wherein assessing the physiological parameter includes estimating a first cardiac output of the patient corresponding to a fixed number multiplied by a change in the constant component of the blood flow in the extracorporeal blood oxygenation circuit.

4. The method of claim 1, wherein the constant is between 1.5 and 2.5.

5. The method of claim 1, wherein assessing the physiological parameter includes. estimating a cardiac output of the patient by multiplying a mean pulsatile flow in the extracorporeal blood oxygenation circuit at a first time by a fixed number to provide a first estimate of the cardiac output of the patient at the first time.

6. The method of claim 5, wherein the fixed number is configured to provide an estimate that is less than an actual cardiac output at the first time.

7. The method of claim 5, further comprising multiplying the mean pulsatile flow in the extracorporeal blood oxygenation circuit at a second time by the fixed number to provide a second estimate of the cardiac output of the patient at the second time.

8. The method of claim 5, wherein the fixed number is selected to provide at least one of the first estimate and the second estimate being less than an actual cardiac output of the patient at the first time and the second time, respectively.

9. The method of claim 1, wherein the physiological parameter is one of a cardiac output CO and a stroke volume SV.

10. The method of claim 1, wherein assessing the physiological parameter includes determining a change in cardiac output of the patient corresponding to a change in one of the constant component in the blood flow in the extracorporeal blood oxygenation circuit and (ii) a pulsatile component in the blood flow in the extracorporeal blood oxygenation circuit.

11. The method of claim 1, wherein an increase in the pulsatile component in the blood. flow in the extracorporeal blood oxygenation circuit corresponds to an increase in the cardiac output of the patient.

12. The method of claim 1, wherein the constant component in the blood flow is a flow rate in the extracorporeal blood oxygenation circuit.

13. The method of claim 1, wherein the constant component in the blood flow is a flow pressure in the extracorporeal blood oxygenation circuit.

14. The method of claim 1, wherein a decrease in the constant component in the blood flow in the extracorporeal blood oxygenation circuit corresponds to an increase in the cardiac output of the patient.

15. An apparatus for monitoring a patient having a circulatory system connected to an extracorporeal blood oxygenation circuit, the extracorporeal blood oxygenation circuit having a blood oxygenator and a pump, wherein the pump imparts at least a partial blood flow through at least one of a portion of the circulatory system and the extracorporeal blood oxygenation circuit, the apparatus comprising:
(a) a controller configured to receive blood flow data of a blood flow in the extracorporeal blood oxygenation circuit, the controller configured to calculate a physiological. parameter of the patient based on one of a constant component of the blood flow and a pulsatile component of the blood flow in the extracorporeal blood oxygenation circuit.

16. The apparatus of claim 15, wherein the controller is configured calculate one of a cardiac output CO and a stroke volume SV of the patient.

17. The apparatus of claim 15, wherein the controller is further configured to calculate a pulsatile component of a cardiac output of the patient at a given frequency as a function of a pulsatile component of the blood flow in the extracorporeal blood oxygenation circuit.

18. The apparatus of claim 15, wherein the controller is further configured to identify, at a given frequency, an increase in a pulsatile component of the cardiac output CO proportional to a measured increase in a pulsatile component of the blood flow in the extracorporeal blood oxygenation circuit.

19. The apparatus of claim 15, wherein the controller is further configured to identify an increase in the pulsatile component in the blood flow in the extracorporeal blood oxygenation circuit which corresponds to an increase in the cardiac output of the patient.

20. The apparatus or claim 15, wherein the controller is further configured to identify a decrease in the constant component in the blood flow in the extracorporeal blood oxygenation circuit which corresponds to an increase in the cardiac output of the patient.
